SEQUENCE LISTING (68 sequences)

SEQ ID NO: 1 - protein, 89 aa, Artificial Sequence
  Description of Artificial Sequence Synthetic polypeptide
LPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 2 - protein, 89 aa, Artificial Sequence
  Description of Artificial Sequence Synthetic polypeptide
LPAPKNLVVSRVTEDSARLSWTAPDAAFDSFLIQYQESEKVGEAIVLTVPGSERSYDLTGLKPGTEYTVSIYGVKGGHRSNPLSAIFTT

SEQ ID NO: 3 - protein, 21 aa, Homo sapiens
MALPVTALLLPLALLLHAARP

SEQ ID NO: 4 - protein, 47 aa, Homo sapiens
TTTPAPRPPTPAPTIASQPLSLRPEACRPAAGGAVHTRGLDFACDIY

SEQ ID NO: 5 - protein, 23 aa, Homo sapiens
IWAPLAGTCGVLLLSLVITLYCK

SEQ ID NO: 6 - protein, 41 aa, Homo sapiens
RGRKKLLYIFKQPFMRPVQTTQEEDGCSCRFPEEEEGGCEL

SEQ ID NO: 7 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 90 aa, Artificial Sequence
  Description of Artificial Sequence Synthetic polypeptide
MLPAPKNLVVSRITEDSARLSWTAPDAAFDSFPIRYIETLIWGEAIWLDVPGSERSYDLTGLKPGTEYTVVIDGVKGGGRSQPLVATFTT

SEQ ID NO: 9 - protein, 90 aa, Artificial Sequence
  Description of Artificial Sequence Synthetic polypeptide
MLPAPKNLVVSRVTEDSARLSWTAPDAAFDSFVIVYSEPDVCGEAIVLTVPGSERSYDLTGLKPGTEYWVRIAGVKGGDFSRPLSAIFTT

SEQ ID NO: 10 - protein, 90 aa, Artificial Sequence
  Description of Artificial Sequence Synthetic polypeptide
MLPAPKNLVVSRVTEDSARLSWIAPDAAFDSFIIVYRENIETGEAIVLTVPGSERSYDLTGLKPGTEYYVQIAGVKGGNISFPLSAIFTT

SEQ ID NO: 11 - protein, 90 aa, Artificial Sequence
  Description of Artificial Sequence Synthetic polypeptide
MLPAPKNLVVSRVTEDSARLSWTAPDAAFDSFPIRYIETLIWGEAIWLDVPGSERSYDLTGLKPGTEYVVVIDGVKGGDHSKPLVATFTT

SEQ ID NO: 12 - protein, 90 aa, Artificial Sequence
  Description of Artificial Sequence Synthetic polypeptide
MLPAPKNLVVSRITEDSARLSWTAPDAAFDSFPIRYIETLIWGEAIWLYVPGSERSYDLTGLKPGTEYTVVISGVKGGESSYPLIAAFTT

SEQ ID NO: 13 - protein, 90 aa, Artificial Sequence
  Description of Artificial Sequence Synthetic polypeptide
MLPAPKNLVVSRVTEDSARLSWTAPDAAFDSFVIVYSEPDVCGEAIVLTVPGSERSYDLTGLKPGTEYWVRIPGVKGGDFFHPLSAIFTT

SEQ ID NO: 14 - protein, 90 aa, Artificial Sequence
  Description of Artificial Sequence Synthetic polypeptide
MLPAPKNLVVSHVTEDSARLSWTAPDAAFDSFIIVYRENIETGEAIVLTVPGSERSYDLTDLKPGTEYYVQIAGVKGGNISFPLSAIFTT

SEQ ID NO: 15 - protein, 90 aa, Artificial Sequence
  Description of Artificial Sequence Synthetic polypeptide
MLPAPKNLVVSRITEDSVRLSWTAPDAAFDSFPIRYIETLIWGEAIWLDVPGSERSYDLTGLKPGTEYVVSIDGVKGGDHSKPLVATFTT

SEQ ID NO: 16 - protein, 90 aa, Artificial Sequence
  Description of Artificial Sequence Synthetic polypeptide
MLPAPKNLVVSRVTEDSARLSWTAPDAAFDSFVIVYSEPDVCGEAIVLTVPGSERSYDLTGLKPGTEYWVRIPGVKGGDFSQPLSAIFTT

SEQ ID NO: 17 - protein, 90 aa, Artificial Sequence
  Description of Artificial Sequence Synthetic polypeptide
MLPAPKNLVVSRITEDSARLSWTAPDAAFDSFPIRYIETLIWGEAIWLDVPGSERSYDLTGLKPGTEYAVVITGVKGGRFSSPLVASFTT

SEQ ID NO: 18 - protein, 90 aa, Artificial Sequence
  Description of Artificial Sequence Synthetic polypeptide
MLPAPKNLVVSSVTEDSARLSWTAPDAAFDSFVIVYSEPDVCGEAIVLTVPGSERSYDLTGLKPGTEYWVRIPGVKGGDFSQPLSAIFTT

SEQ ID NO: 19 - protein, 90 aa, Artificial Sequence
  Description of Artificial Sequence Synthetic polypeptide
MLPAPKNLVVSRVTEDSARLSWTAPDAAFDSFIIVYRENIETGEAIVLTVPGSERSYDLTGLKPGTEYYVQIAGVKGGNISFPLSAIFTT

SEQ ID NO: 20 - protein, 90 aa, Artificial Sequence
  Description of Artificial Sequence Synthetic polypeptide
MLPAPKNLVVSRITEDSARLSWTAPDAAFDSFPIRYIETLIWGEAIWLDVPGSERSYDLTGLKPGTEYTVVIDGVKGGGRSQPLFAQFTT

SEQ ID NO: 21 - protein, 90 aa, Artificial Sequence
  Description of Artificial Sequence Synthetic polypeptide
MLPAPKNLVVSRITEDSARLSWTAPDAAFDSFPIRYIETLIWGEAIWLDVPGSERSYDLTGLKPGTEYVVVISGVKGGWESTPLVAPFTT

SEQ ID NO: 22 - protein, 90 aa, Artificial Sequence
  Description of Artificial Sequence Synthetic polypeptide
MLPAPKNLVVSRITEDSARLSWTAPDAAFDSFWIRYVERLVWGEAIHLHVPGSERSYDLTGLKPGTEYVVVISGVKGGWESTPLVAPFTT

SEQ ID NO: 23 - protein, 90 aa, Artificial Sequence
  Description of Artificial Sequence Synthetic polypeptide
MLPAPKNLVVSRITEDSARLSWTAPDAAFDSFPIRYVERIVWGEAIWLHVPGSERSYDLTGLKPGTEYVVVISGVKGGWESTPLVAPFTT

SEQ ID NO: 24 - protein, 90 aa, Artificial Sequence
  Description of Artificial Sequence Synthetic polypeptide
MLPAPKNLVVSRITEDSARLSWTAPDAAFDSFPIRYIETLIWGEAIWLYVPGSERSYDLTGLKPGTEYTVVIDGVKGGGRSQPLVASFTT

SEQ ID NO: 25 - protein, 90 aa, Artificial Sequence
  Description of Artificial Sequence Synthetic polypeptide
MLPAPKNLVVSRITEDSARLSWTAPDAAFDSFPIRYIETLIWGEAIWLDVPGSERSYDLTGLKPGTEYTVVIGGVKGGHNSWPLSAKFTT

SEQ ID NO: 26 - protein, 90 aa, Artificial Sequence
  Description of Artificial Sequence Synthetic polypeptide
MLPAPKNLVVSRITEDSARLSWTAPDAAFDSFWIRYVERLVWGEAIHLHVPGSERSYDLTGLKPGTEYVVVISGVKGGEQSHPLYATFTT

SEQ ID NO: 27 - protein, 90 aa, Artificial Sequence
  Description of Artificial Sequence Synthetic polypeptide
MLPAPKNLVVSRVTEDSARLSWTAPDAAFDSFPIRYIETLIWGEAIWLQVPGSERSYDLTGLKPGTEYVVVISGVKGGWESKPLIAAFTT

SEQ ID NO: 28 - protein, 90 aa, Artificial Sequence
  Description of Artificial Sequence Synthetic polypeptide
MLPAPKNLVVSRITEDSARLSWTAPDAAFDSFPIRYIETLIWEEAIWLDVPGSERSYDLTGLKPGTEYTVVIDGVKGGGRSQPLVASFTT

SEQ ID NO: 29 - protein, 90 aa, Artificial Sequence
  Description of Artificial Sequence Synthetic polypeptide
MLPAPKNLVVSRITEDSARLSWTAPDAAFDSFPIRYIETLIWGEAIWLYVPGSERSYDLTGLKPGTEYVVVISGVKGGEQSHPLYATFTT

SEQ ID NO: 30 - protein, 90 aa, Artificial Sequence
  Description of Artificial Sequence Synthetic polypeptide
MLPAPKNLVVSRITEDSARLSWTAPDAAFDSFPIRYIETLIWGEAIWLFVPGSERSYDLTGLKPGTEYVVVISGVKGGEQSHPLYATFTT

SEQ ID NO: 31 - protein, 90 aa, Artificial Sequence
  Description of Artificial Sequence Synthetic polypeptide
MLPAPKNLVVSRITEDSARLSWTAPDAAFDSFTIKYIERATWGEAIWLNVPGSERSYDLTGLKPGTEYVVLINGVKGGPESWPLIAYFTT

SEQ ID NO: 32 - protein, 90 aa, Artificial Sequence
  Description of Artificial Sequence Synthetic polypeptide
MLPAPKNLVVSRITEDSARLSWTAPDAAFDSFPIRYIETLIWGEAIWLHVPGSERSYDLTGLKPGTEYVVVISGVKGGEQSHPLYATFTT

SEQ ID NO: 33 - protein, 90 aa, Artificial Sequence
  Description of Artificial Sequence Synthetic polypeptide
MLPAPKNLVVSRVTEDSARLSWTAPDAAFDSFPIRYIETLIWGEAIWLDVPGSERSYDLTGLKPGTEYVVVISGVKGGEQSHPLYATFTT

SEQ ID NO: 34 - protein, 90 aa, Artificial Sequence
  Description of Artificial Sequence Synthetic polypeptide
MLPAPKNLVVSRVTEDSARLSWTAPDAAFDSFTIKYIERATWGEAIWLNVPGSERSYDLTGLKPGTEYVVLINGVKGGPESWPLWASFTT

SEQ ID NO: 35 - protein, 90 aa, Artificial Sequence
  Description of Artificial Sequence Synthetic polypeptide
MLPAPKNLVVSRVTEDSARLSWTAPDAAFDSFRIRYVEVIAWGEAIWLVVPGSERSYDLTGLKPGTEYVVVIDGVKGGKTSIPLIAHFTT

SEQ ID NO: 36 - protein, 90 aa, Artificial Sequence
  Description of Artificial Sequence Synthetic polypeptide
MLPAPKNLVVSRITEDSARLSWTAPDAAFDSFTIKYIERATWGEAIWLNVPGSERSYDLTGLKPGTEYVVLINGVKGGPESWPLIAHFTT

SEQ ID NO: 37 - protein, 90 aa, Artificial Sequence
  Description of Artificial Sequence Synthetic polypeptide
MLPAPKNLVVSRITEDSARLSWTAPDAAFDSFPIRYIETLIWGEAIWLDVPGSERSYDLTGLKPGTEYVVVISGVKGGEQSHPLYATFTT

SEQ ID NO: 38 - protein, 90 aa, Artificial Sequence
  Description of Artificial Sequence Synthetic polypeptide
MLPAPKNLVVSRITEDSARLSWTAPDAAFDSFPIRYIETLIWGEAIWLDVPGSERSYDLTGLKPATEYVVVITGVKGGRKSYPLVAEFTT

SEQ ID NO: 39 - protein, 90 aa, Artificial Sequence
  Description of Artificial Sequence Synthetic polypeptide
MLPAPKNLVVSRITEDSARLSWTAPDAAFDSFPIRYIETLIWGEAIWLDVPGSERSYDLTGLKPGTEYLVVISGVKGGRDSQPLITHFTT

SEQ ID NO: 40 - protein, 90 aa, Artificial Sequence
  Description of Artificial Sequence Synthetic polypeptide
MLPAPKNLVVSRVTEDSARLSWTAPDAAFDSFTIKYIERATWGEAIWLNVPGSERSYDLTGLKPGTEYVVLINGVKGGPESWPLIAYFTT

SEQ ID NO: 41 - protein, 90 aa, Artificial Sequence
  Description of Artificial Sequence Synthetic polypeptide
MLPAPKNLVVSRITEDSARLSWTAPDAAFDSFWIRYVERLVWGEAIHLHVPGSERSYDLTGLKPGTEYVVSIDGVKGGDHSKPLVATFTT

SEQ ID NO: 42 - protein, 90 aa, Artificial Sequence
  Description of Artificial Sequence Synthetic polypeptide
MLPAPKNLVVSRVTEDSARLSWTAPDAAFDSFVIQYIERLRWGEAITLGVPGSERSYDLTGLKPGTEYVVPISGVKGGRTSTPLIASFTT

SEQ ID NO: 43 - protein, 90 aa, Artificial Sequence
  Description of Artificial Sequence Synthetic polypeptide
MLPAPKNLVVSRVTEDSARLSWTTPDAAFDSFTIKYIERATWGEAIWLNVPGSERSYDLTGLKPGTEYVVLINGVKGGPESWPLIAYFTT

SEQ ID NO: 44 - protein, 90 aa, Artificial Sequence
  Description of Artificial Sequence Synthetic polypeptide
MLPAPKNLVVSRVTEDSARLSWTAPDAAFDSFIIGYIEQIVWGEAIHLNVPGSERSYDLTGLKPGTEYVVIIRGVKGGSFSEPLVAPFTT

SEQ ID NO: 45 - protein, 153 aa, Artificial Sequence
  Description of Artificial Sequence Synthetic polypeptide
RGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 21 aa, Artificial Sequence
  Description of Artificial Sequence Synthetic peptide
MEHSTFLSGLVLATLLSQVSP

SEQ ID NO: 47 - protein, 22 aa, Artificial Sequence
  Description of Artificial Sequence Synthetic peptide
MQSGTHWRVLGLCLLSVGVWGQ

SEQ ID NO: 48 - protein, 22 aa, Artificial Sequence
  Description of Artificial Sequence Synthetic peptide
MLLLVTSLLLCELPHPAFLLIP

SEQ ID NO: 49 - protein, 23 aa, Artificial Sequence
  Description of Artificial Sequence Synthetic peptide
MGNSCYNIVATLLLVLNFERTRS

SEQ ID NO: 50 - protein, 27 aa, Artificial Sequence
  Description of Artificial Sequence Synthetic peptide
FWVLVVVGGVLACYSLLVTVAFIIFWV

SEQ ID NO: 51 - protein, 22 aa, Artificial Sequence
  Description of Artificial Sequence Synthetic peptide
MALIVLGGVAGLLLFIGLGIFF

SEQ ID NO: 52 - protein, 26 aa, Artificial Sequence
  Description of Artificial Sequence Synthetic peptide
IYLIIGICGGGSLLMVFVALLVFYIT

SEQ ID NO: 53 - protein, 26 aa, Artificial Sequence
  Description of Artificial Sequence Synthetic peptide
NLGSVYIYVLLIVGTLVCGIVLGFLF

SEQ ID NO: 54 - protein, 245 aa, Artificial Sequence
  Description of Artificial Sequence Synthetic polypeptide
QSLEESGGRLVTPGTPLTLTCTVSGIDLSTSVMGWVRQAPGKGLESIGFIYTNVNTYYASWAKGRFTISRTSTTVDLKITSPTTGDTATYFCARAVYAGAMDLWGQGTLVTVSSGGGGSGGGGSGGGGSGGGGSDVVMTQTPASVSGPVGGTVTIKCQASERIYSNLAWYQQKPGQPPKLLIYKASTLASGVSSRFKGSGSGTEFTLTIRDLECADAATYSCQYTSYGSGYVGTFGGGTEVVVEG

SEQ ID NO: 55 - protein, 243 aa, Artificial Sequence
  Description of Artificial Sequence Synthetic polypeptide
DVVMTQTPASVSGPVGGTVTIKCQASERIYSNLAWYQQKPGQPPKLLIYKASTLASGVSSRFKGSGSGTEFTLTIRDLECADAATYSCQYTSYGSGYVGTFGGGTEVVVEGGGGGSGGGGSGGGGSGGGGSLEESGGRLVTPGTPLTLTCTVSGIDLSTSVMGWVRQAPGKGLESIGFIYTNVNTYYASWAKGRFTISRTSTTVDLKITSPTTGDTATYFCARAVYAGAMDLWGQGTLVTVSS

SEQ ID NO: 56 - RNA, 20 nt, Artificial Sequence
  Description of Artificial Sequence Synthetic oligonucleotide
gcugguacacggcaggguca

SEQ ID NO: 57 - RNA, 20 nt, Artificial Sequence
  Description of Artificial Sequence Synthetic oligonucleotide
gagaaucaaaaucggugaau

SEQ ID NO: 58 - RNA, 20 nt, Artificial Sequence
  Description of Artificial Sequence Synthetic oligonucleotide
ccggugccucgcucuguaga

SEQ ID NO: 59 - RNA, 19 nt, Artificial Sequence
  Description of Artificial Sequence Synthetic oligonucleotide
acucucucuuucugccugg

SEQ ID NO: 60 - RNA, 19 nt, Artificial Sequence
  Description of Artificial Sequence Synthetic oligonucleotide
agagccacguguccccucg

SEQ ID NO: 61 - RNA, 20 nt, Artificial Sequence
  Description of Artificial Sequence Synthetic oligonucleotide
ucuccuggaauauucugccg

SEQ ID NO: 62 - DNA, 3396 bp, Homo sapiens
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggac

SEQ ID NO: 63 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 64 - protein, 157 aa, Artificial Sequence
  Description of Artificial Sequence Synthetic polypeptide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTKDEL

SEQ ID NO: 65 - DNA, 474 bp, Artificial Sequence
  Description of Artificial Sequence Synthetic polynucleotide
atgtatcgtatgcaactgctgagctgcatcgctttatctttagctttagtgaccaattccgcccccaccagcagcagcaccaagaagacacagctgcagctggagcatttactgctggatttacagatgattttaaacggcatcaacaactacaaaaaccccaagctgacaaggatgctgaccttcaagttctacatgcccaagaaggccaccgagctgaagcatttacagtgtttagaggaggagctgaagcctttagaggaggtgctgaatttagcccagagcaagaacttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaaccaaggacgagctgtga

SEQ ID NO: 66 - protein, 12 aa, Artificial Sequence
  Description of Artificial Sequence Synthetic peptide
GSGSGSGSGSGS

SEQ ID NO: 67 - protein, 9 aa, Unknown
  Description of Unknown “LAGLIDADG” family peptide motif sequence
LAGLIDADG

SEQ ID NO: 68 - protein, 4 aa, Artificial Sequence
  Description of Artificial Sequence Synthetic peptide
KDEL

(19) United States
(12) Patent Application Publication
(10) Pub. No.: US 2019/0175651 A1
(43) Pub. Date: Jun. 13, 2019

(54) IMMORTALIZED CAR-T CELLS GENETICALLY MODIFIED TO ELMINATE T-CELL RECEPTOR AND BETA 2-MICROGLOBULIN EXPRESSION

(71) Applicant: Janssen Biotech, Inc., Horsham, PA (US)

(72) Inventors: John Lee, Spring House, PA (US); Jill Mooney, Spring House, PA (US); Michael Naso, Spring House, PA (US)

(73) Assignee: Janssen Biotech, Inc., Horsham, PA (US)

(21) Appl. No.: 16/215,716

(22) Filed: Dec. 11, 2018

Related U.S. Application Data
(60) Provisional application No. 62/598,032, filed on Dec. 13, 2017.

(51) Int. Cl. A61K 35/17; C07K 14/78; C07K 14/47; C07K 14/705; C07K 14/715; A61P 35/00; C12N 15/85; C12N 15/90

(52) U.S. Cl. CPC A61K 35/17; C07K 14/78; C07K 14/4748; C12N 15/902; C07K 14/7151; A61P 35/00; C12N 15/8509; C07K 14/70578

(57) ABSTRACT

The present invention pertains to engineered immortalized T-cell lines, method for their preparation and their use as medicament, particularly for immunotherapy. The engineered immortalized T-cell lines of the invention are characterized in that the expression of endogenous T-cell receptors (TCRs) and beta 2-microglobulin (B2M) is inhibited, e.g., by using an endonuclease able to selectively inactivate the TCR and B2M genes in order to render the immortalized T-cells non-alloreactive. In addition, expression of immunosuppressive polypeptide can be performed on those engineered immortalized T-cells in order to prolong the survival of these T-cells in host organisms. Such engineered immortalized T-cells are particularly suitable for allogeneic transplantations, especially because it reduces both the risk of rejection by the host's immune system and the risk of developing graft versus host disease. The invention opens the way to standard and affordable adoptive immunotherapy strategies using immortalized T-cells for treating cancer, infections and auto-immune diseases.

### SEQUENCE LISTING

The instant application contains a Sequence Listing which has been submitted electronically in ASCII format and is hereby incorporated by reference in its entirety. Said ASCII copy, created on Nov. 20, 2018, is named JBI5146USNP1_SL.txt and is 65,915 bytes in size.

### FIELD OF THE INVENTION

The present invention pertains to engineered immortalized T-cell lines expressing a chimeric antigen receptor (CAR), method for their preparation and their use as medicament, particularly for immunotherapy. The engineered immortalized CAR T-cells of the invention are characterized in that the expression of endogenous T-cell receptors (TCRs) and beta 2-microglobulin (B2M) is inhibited, e.g., by using an endonuclease able to selectively inactivate the TCR and B2M genes in order to render the immortalized CAR T-cells non-alloreactive. The engineered immortalized CAR T-cell lines are particularly suitable for allogeneic transplantations, especially because it reduces both the risk of rejection by the host's immune system and the risk of developing graft versus host disease. The invention opens the way to standard and affordable adoptive immunotherapy strategies using T-cells for treating cancer, infections and auto-immune diseases.

### BACKGROUND OF THE INVENTION

Adoptive immunotherapy, which involves the transfer of autologous antigen-specific T-cells generated ex vivo, is a promising strategy to treat viral infections and cancer. The T-cells used for adoptive immunotherapy can be generated either by expansion of antigen-specific T-cells or redirection of T-cells through genetic engineering (Park, T. S., S. A. Rosenberg, et al. (2011). “Treating cancer with genetically engineered T cells.” Trends Biotechnol 29(11): 550-7).

Novel specificities in T-cells have been successfully generated through the genetic transfer of transgenic T-cells receptors or chimeric antigen receptors (CARs) (Jena, B., G. Dotti, et al. (2010). “Redirecting T cell specificity by introducing a tumor-specific chimeric antigen receptor.” Blood 116(7): 1035-44). CARs are synthetic receptors consisting of a targeting moiety that is associated with one or more signaling domains in a single fusion molecule. In general, the binding moiety of a CAR consists, for example, of an antigen-binding domain of a single-chain antibody (scFv), comprising the light and variable fragments of a monoclonal antibody joined by a flexible linker. The signaling domains for first generation CARs are derived from the cytoplasmic region of the CD3zeta or the Fc receptor gamma chains. First generation CARs have been shown to successfully redirect T-cell cytotoxicity. However, they failed to provide prolonged expansion and anti-tumor activity in vivo. Signaling domains from co-stimulatory molecules including CD28, OX-40 (CD134), and 4-1BB (CD137) have been added alone (second generation) or in combination (third generation) to enhance survival and increase proliferation of CAR modified T-cells. CARs have successfully allowed T-cells to be redirected against antigens expressed at the surface of tumor cells from various malignancies including lymphomas and solid tumors (Jena, Dotti et al. 2010).

The current protocol for treatment of patients using adoptive immunotherapy is based on autologous cell transfer. In this approach, T lymphocytes are recovered from patients, genetically modified or selected ex vivo, cultivated in vitro in order to amplify the number of cells if necessary, and finally infused into the patient. In addition to lymphocyte infusion, the host may be manipulated in other ways that support the engraftment of the T cells or their participation in an immune response, for example pre-conditioning (with radiation or chemotherapy) and administration of lymphocyte growth factors (such as IL-2). Each patient receives an individually fabricated treatment, using the patient's own lymphocytes (i.e. an autologous therapy). Autologous therapies face substantial technical and logistic hurdles to practical application, their generation requires expensive dedicated facilities and expert personnel, they must be generated in a short time following a patient's diagnosis, and in many cases, pretreatment of the patient has resulted in degraded immune function, such that the patient's lymphocytes may be poorly functional and present in very low numbers. Because of these hurdles, each patient's autologous cell preparation is effectively a new product, resulting in substantial variations in efficacy and safety.

Ideally, one would like to use a standardized therapy in which allogeneic therapeutic cells could be pre-manufactured, characterized in detail, and available for immediate administration to patients. By allogeneic it is meant that the cells are obtained from individuals belonging to the same species but are genetically dissimilar. However, the use of allogeneic cells presently has many drawbacks. In immune-competent hosts allogeneic cells are rapidly rejected, a process termed host versus graft rejection (HvG), and this substantially limits the efficacy of the transferred cells. In immune-incompetent hosts, allogeneic cells are able to engraft, but their endogenous T-cells receptors (TCR) specificities may recognize the host tissue as foreign, resulting in graft versus host disease (GvHD), which can lead to serious tissue damage and death.

Thus, a need in the art remains to develop methods and reagents that circumvent the time, expense to manufacture, and risk of rejection for patient-specific T-cell products.

### SUMMARY OF THE INVENTION

The present invention provides engineered immortalized T cell lines suitable for immunotherapy purposes. The present invention more particularly provides T cell lines with no expression of certain effector molecules important for immune recognition and histocompatibility.

In one general aspect, the invention relates to an engineered immortalized T cell line expressing a CAR, comprising an extracellular domain, a transmembrane domain, and an intracellular domain, the extracellular domain comprising an antigen binding region. The engineered immortalized T cell line of the invention does not express at least one endogenous T-cell receptor (TCR) and does not express beta 2-microglobulin (B2M).

In one embodiment, the expression of the at least one endogenous TCR and B2M is eliminated by gene knockout. In a specific embodiment, the engineered immortalized T cell line of the invention does not express TCR-alpha. In another embodiment, the engineered immortalized T cell line of the invention does not express KIR3DL2.

In another embodiment, the engineered immortalized T cell line of the invention does not express B2M.

In another embodiment, the engineered immortalized T cell line of the invention comprises a CAR comprising an extracellular domain binding specifically to a tumor associated antigen. In a specific embodiment, the engineered immortalized T cell line can comprise a CAR comprising an extracellular domain binding specifically to BCMA.

In another embodiment, the engineered immortalized T cell line can comprise a CAR comprising an extracellular domain binding specifically to a fibronectin type III (FN3) domain.

In another general aspect, the invention relates to an engineered TALL-104 cell line expressing a CAR, comprising an extracellular domain, a transmembrane domain, and an intracellular domain, the extracellular domain comprising an antigen binding region. The engineered TALL-104 cell line of the invention does not express at least one endogenous T-cell receptor (TCR) and does not express beta 2-microglobulin (B2M).

In one embodiment, the expression of the at least one endogenous TCR and B2M is eliminated by gene knockout. In a specific embodiment, the engineered TALL-104 cell line of the invention does not express TCR-alpha. In another embodiment, the engineered TALL-104 cell line of the invention does not express KIR3DL2.

In another embodiment, the engineered TALL-104 line of the invention does not express B2M.

In another embodiment, the engineered TALL-104 cell line of the invention comprises a CAR comprising an extracellular domain binding specifically to a tumor associated antigen. In a specific embodiment, the engineered TALL-104 line can comprise a CAR comprising an extracellular domain binding specifically to BCMA.

In another embodiment, the engineered TALL-104 cell line can comprise a CAR comprising an extracellular domain binding specifically to a fibronectin type III (FN3) domain.

In another general aspect, the invention relates to an engineered TALL-104 cell line expressing a CAR, comprising:
(a) a signal peptide having an amino acid sequence of SEQ ID NO: 3;
(b) an extracellular domain comprising an FN3 domain having an amino acid sequence of any one of SEQ ID NOs: 8-44;
(c) a hinge region having an amino acid sequence of SEQ ID NO: 4;
(d) a transmembrane domain having an amino acid sequence of SEQ ID NO: 5; and
(e) an intracellular signaling domain comprising a co-stimulatory domain having an amino acid sequence of SEQ ID NO: 6, and a primary signaling domain having an amino acid sequence of SEQ ID NO: 7;
   wherein the cell line does not express TRCA, KIR3DL2 and B2M.

In another general aspect, the invention relates to an engineered TALL-104 cell line expressing a CAR, comprising:
(a) an extracellular domain comprising an scFv having an amino acid sequence of any one of SEQ ID NOs: 54 and 55;
(b) a hinge region having an amino acid sequence of SEQ ID NO: 4;
(c) a transmembrane domain having an amino acid sequence of SEQ ID NO: 5; and
(d) an intracellular signaling domain comprising a co-stimulatory domain having an amino acid sequence of SEQ ID NO: 6, and a primary signaling domain having an amino acid sequence of SEQ ID NO: 7.
   wherein the cell line does not express TRCA, KIR3DL2 and B2M.

In another general aspect, the invention also relates to an in vitro method of generating an engineered immortalized T cell line expressing a CAR, comprising the steps of:
a. providing an immortalized T cell line;
b. inhibiting the expression of at least one endogenous T cell receptor and B2M; and
c. introducing a polynucleotide that encodes a CAR into the immortalized T cell.

In one embodiment, step b occurs before step c.

In another embodiment step c occurs before step b.

In another embodiment, step b is performed by using an endonuclease. In a specific embodiment, the RNA-guided endonuclease is a TAL-nuclease, meganuclease, zing-finger nuclease (ZFN), or Cas9.

In another embodiment, the polynucleotide that encodes a CAR is introduced into the immortalized T cell by electroporation.

In another embodiment, the polynucleotide that encodes a CAR is introduced into the immortalized T cell via a viral-based gene transfer system. In specific embodiments, the viral-based gene transfer system comprises a retroviral vector, adenoviral vector, adeno-associated viral vector, or lentiviral vector.

In another general aspect, the invention relates to pharmaceutical compositions comprising engineered immortalized T cells of the invention.

In another general aspect, the invention relates to a method of treating a cancer in a subject in need thereof, comprising administering to the subject a therapeutically effective amount of a pharmaceutical composition of the invention. In a preferred embodiment, the cancer is multiple myeloma.

In another general aspect, the invention relates to a method of producing a pharmaceutical composition, comprising combining the engineered immortalized T cell lines of the invention with a pharmaceutically acceptable carrier to obtain the pharmaceutical composition.

Other aspects, features and advantages of the invention will be apparent from the following disclosure, including the detailed description of the invention and its preferred embodiments and the appended claims.

### BRIEF DESCRIPTION OF THE DRAWINGS

FIGS. 1A and 1B. Flow cytometry analysis of CRISPR-Cas9-mediated gene editing of HLA Class I (A) and TCR (B) in TALL-104 cells. TALL-104 cells electroporated with Beta 2 Microglubulin (B2M) and TCRa ribonucleoprotein (RNP) complexes were re-suspended in FACS stain buffer and antibodies were added according to manufacturer's instructions. Cells were incubated in the dark at 4° C. for 45 mins and data was collected on a BD FACS Calibur flow cytometer.

FIGS. 2A and 2B. Purification of B2M/HLA-1 (A) and TCR (B) knockout TALL-104 cell populations. TALL-104 cells previously electroporated with either B2M or TCRa ribonucleoprotein (RNP) complexes were labeled with PE anti-B2M (A) or PE anti-CD3 antibodies. Antibody-labeled cells were incubated with anti-PE microbeads and passed through an LS column attached to a QuadroMACS separator. B2M and CD3-KO cell sub-populations collected in the eluate were centrifuged and re-suspended in Compete TALL-104 cell media and cultures at 37° C.

FIG. 3A-3F. Expression and detection of CARS targeting BCMA or FN3 domains on TALL-104 cells by flow cytometry. TALL-104 BCMA-CAR Cells (A) and TALL-104 anti-FN3 domain CAR Cells (B) were measured for binding of polyclonal anti-FN3 domain antibody and conjugated FN3 domain respectively to cells compared to binding to Mock (no mRNA) electroporated control cells (grey) using BD Biosciences FACSCalibur. Data were analyzed using FlowJo version 10 to gate on cell population by scatter and positive binding by Alexa647 or APC intensity.

FIG. 4A-4C: TALL-104 CAR-expressing cell killing of BCMA target cells. TALL-104 anti-FN3 domain CAR Cells were assessed for killing of BCMA target cells at 20 hours (A) and 40 hours (B) after co-incubating with a BCMA-specific or non-targeted control (NT) FN3 domain. TALL-104 BCMA-CAR Cells (C) were assessed for killing of BCMA target cells at 20 hours after co-incubating cells.

FIG. 5: TALL-104 cells were transduced with lentivirus encoding the human TERT gene and EGFP. Cells were sorted for EGFP expression and then allowed to expand in TALL-104 culture conditions. Growth profile after wild-type non-transduced cells had stopped proliferating in culture is displayed.

FIG. 6: hTERT positive TALL-104 cells were transduced with lentivirus p102 and maintained in the absence of exogenous IL-2.

### DETAILED DESCRIPTION OF THE INVENTION

### Definitions

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

There are three general types of cell cultures: (1) Primary—derived from human or animal tissues and organs (pluripotent stem cells and tissue-specific progenitors are included in this category), (2) Immortalized (or continuous)—derived from primary cells which have been engineered to divide and proliferate indefinitely in culture (these cells retain many characteristics of normal primary cells, such as contact-inhibition of growth in the case of adherent fibroblasts), and (3) Transformed—derived from cancerous tissues or oncogenically transformed in vitro by cancer-inducing viruses (these cells do not resemble normal primary cells and behave like tumor cells. Transformed cells exhibit a loss of contact-inhibition, growth factor-independence or reduced requirement for soluble growth factors and serum, and anchorage (ECM)-independent growth (Flint S J, Enquist L W, Racaniello V R. and Skalka A M (2004). Virus cultivation, detection, and genetics, In Principles of Virology: Molecular Biology. Pathogenesis, and Control of Animal Viruses. 2^{nd} Edition (ASM Press, Washington, D.C.). pp 26-62). For most biomedical and pharmaceutical research and development applications (e.g., in vitro efficacy and toxicity testing of pharmacological drug candidates), it is generally desirable to use a cellular background that closely recapitulates normal physiological conditions. While primary cultures most closely resemble the normal tissue microenvironment, there are significant difficulties in obtaining these cells from human or animal tissues and complex regulatory requirements (e.g., Institutional Animal Care & Usage Committees; Human Subjects Research-Institutional Review Boards), and the general difficulties associated with maintaining and growing primary cells in vitro (growth factor- and stromal-dependence), make it difficult to use these cells for most applications. Primary cells have a finite doubling-capacity (usually 40-60 replication cycles) before they undergo crisis and senescence (Weinberg R A (2007). Eternal life: cell immortalization and tumorigenesis, In The Biology of Cancer (Garland Science, New York), pp 357-398). The use of primary cultures can also introduce significant reproducibility errors, as these cells must be continually re-isolated to conduct multiple experiments.

Therefore, as used herein the term “immortalized” or “continuous” with regard to the cellular characteristics of cell lines derived from primary cells refers to a T-lymphocytes (or T cells) engineered to divide and proliferate indefinitely in culture. These cells retain many characteristics of normal primary cells, such as, e.g., contact-inhibition of growth in the case of adherent cells and IL-2 dependence.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A“cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC # CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Bel3, CCRF-CEM, CML-T, DND-41, DU.528, EU-9, HD-Mar, HPB-ALL, H-SB2, HT-1, JK-TI, Jurkat, Karpas 45, KE-37, KOPT-Ki, K-TI, L-KAW, Loucy, MAT, MOLT-1, MOLT 3, MOLT-4, MOLT 13, MOLT-16, MT-1, MT-ALL, Pl2/Ichikawa, Peer, PER0117, PER-255, PF-382, PFI-285, RPMI-8402, ST-4, SUP-TI to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

The term “chimeric antigen receptors (CARs)” as used herein may be referred to as artificial T-cell receptors, chimeric T-cell receptors, or chimeric immune-receptors, for example, and encompass engineered receptors that graft an artificial specificity onto a particular immune effector cell. The CARs may be employed to impart the specificity of a monoclonal antibody onto a T cell, thereby allowing a large number of specific T cells to be generated, for example, in use for adoptive cell therapy. In specific embodiments, the CARs direct specificity of the cell to a tumor associated antigen, for example. In some embodiments, the CARs comprise an intracellular activation domain, a transmembrane domain and an extracellular domain comprising a tumor associated antigen binding region. In particular aspects, CARs comprise fusions of single-chain variable fragments (scFv) derived from monoclonal antibodies, fused to CD3-zeta transmembrane and endodomain. In other aspects, CARs comprise fusions of fibronectin type III domains, fused to CD3-zeta transmembrane and endodomain. The specificity of other CARs designs may be derived from ligands of receptors (e.g., peptides) or from Dectins. In particular embodiments, one can target malignant B cells by redirecting the specificity of T cells using a chimeric immunoreceptor specific for the B-lineage molecule, BCMA. In certain cases, the CARs comprise domains for additional co-stimulatory signaling, such as CD3-zeta, FcR, CD27, CD28, CD137, DAP 10, and/or OX40. In some cases molecules can be co-expressed with the CAR.

These include co-stimulatory molecules, reporter genes for imaging (e.g., for positron emission tomography), gene products that conditionally ablate the T cells upon addition of a pro-drug, homing receptors, cytokines, and cytokine receptors.

As used herein, the term “extracellular domain,” refers to the part of a CAR that is located outside of the cell membrane and is capable of binding to an antigen, target or ligand.

As used herein, the term “transmembrane domain” refers to the portion of a CAR that extends across the cell membrane and anchors the CAR to cell membrane.

As used herein, the term “intracellular signaling domain” refers to the part of a CAR that is located inside of the cell membrane and is capable of transducing an effector signal.

The term “express” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed T cell receptor and beta-2 microbulin can be anchored to the T cell membrane.

The term “T cell receptor (TCR)” as used herein refers to a protein receptor on T cells that is composed of a heterodimer of an alpha (α) and beta (β) chain, although in some cells the TCR consists of gamma and delta (γ/δ) chains. In embodiments of the invention, the TCR may be modified on any cell comprising a TCR, including a helper T cell, a cytotoxic T cell, a memory T cell, regulatory T cell, natural killer T cell, and gamma delta T cell, for example.

“Beta-2 microglobulin”, also known as “B2M”, is the light chain of MHC class I molecules, and as such an integral part of the major histocompatibility complex. In humans, B2M is encoded by the b2m gene which is located on chromosome 15, opposed to the other MHC genes which are located as gene cluster on chromosome 6. The human protein is composed of 119 amino acids and has a molecular weight of 11.8 Kilodaltons. Mice models deficient for beta-2 microglobulin have shown that B2M is necessary for cell surface expression of MHC class I and stability of the peptide binding groove. It was further shown that haemopoietic transplants from mice that are deficient for normal cell-surface MHC I expression are rejected by NK1.1+ cells in normal mice because of a targeted mutation in the beta-2 microglobulin gene, suggesting that deficient expression of MHC I molecules renders marrow cells susceptible to rejection by the host immune system (Bix M. et al (1991). “Rejection of class I MHC-deficient haemopoietic cells by irradiated MHC-matched mice.” Nature 349(6307):329-31).

As used herein, the term “BCMA” refers to a B cell maturation antigen protein (also referred to as TNFRSF17, BCM or CD269), a tumor necrosis factor receptor (TNFR) family member that is expressed on plasma cells and on mature B cells. For example, a human BCMA is a 184 amino acid-long protein encoded by a primary mRNA transcript 994 nucleotides long (NM_001192.2). The amino acid sequence of human BCMA is represented in GenBank Accession No. NP_001183.2. As used herein, the term “BCMA” includes proteins comprising mutations, e.g., point mutations, fragments, insertions, deletions and splice variants of full length wild type BCMA. The term “BCMA” also encompasses post-translational modifications of the BCMA amino acid sequence. Post-translational modifications include, but are not limited to, N- and O-linked glycosylation.

As used herein, the term “fibronectin type III domain” or “FN3 domain” refers to a domain occurring frequently in proteins including fibronectins, tenascin, intracellular cytoskeletal proteins, cytokine receptors and prokaryotic enzymes (Bork and Doolittle, PNAS USA 89:8990-8994, 1992; Meinke et al., J Bacteriol 175:1910-1918, 1993; Watanabe et al., J Biol Chem 265:15659-15665, 1990), or a derivative thereof. Exemplary FN3 domains are the 15 different FN3 domains present in human tenascin C, the 15 different FN3 domains present in human fibronectin (FN), and non-natural synthetic FN3 domains, for example, in U.S. Pat. No. 8,278,419. Individual FN3 domains are referred to by domain number and protein name, e.g., the 3^{rd} FN3 domain of tenascin (TN3), or the 10^{th} FN3 domain of fibronectin (FN10).

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention.

As used herein, the term “subject” refers to an animal, and preferably a mammal. According to particular embodiments, the subject is a mammal including a non-primate (e.g., a camel, donkey, zebra, cow, pig, horse, goat, sheep, cat, dog, rat, rabbit, guinea pig or mouse) or a primate (e.g., a monkey, chimpanzee, or human). In particular embodiments, the subject is a human.

The term “cancer” as used herein means any disease, condition, trait, genotype or phenotype characterized by unregulated cell growth or replication as is known in the art. A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive, and is also referred to as a neoplasm. Tumors can be either benign (not cancerous) or malignant. The compositions and methods described herein are useful for treatment of cancer and tumor cells, i.e., both malignant and benign tumors. Thus, in various embodiments of the methods and compositions described herein, the cancer can include, without limitation, heme cancers, lymphomas, breast cancer, lung cancer, prostate cancer, colorectal cancer, esophageal cancer, stomach cancer, bladder cancer, pancreatic cancer, kidney cancer, cervical cancer, liver cancer, ovarian cancer, and testicular cancer.

As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein, the terms “treat,” “treating,” and “treatment” are all intended to refer to an amelioration or reversal of at least one measurable physical parameter related to a cancer or autoimmunity, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

### General Embodiments of the Invention

Chimeric antigen receptors (CARs) are designed for adoptive immunotherapy by connecting an extracellular antigen-binding domain to a transmembrane domain and an intracellular signaling domain (endodomain). It is a useful anti-tumor approach to eradicate tumor cells by adoptive transfer of T cells expressing chimeric antigen receptors to recognize specific antigens presented on tumor cells and activate T cells to specifically lyse these tumor cells. A critical aspect of this CAR strategy is the selection of target epitopes that are specifically or selectively expressed on tumors, are present on all tumor cells, and are membrane epitopes not prone to shed or modulate from the cell surface. However, ideally the CAR-T cells would be able to be used as a universal reagent or drug suitable for any mammalian (such as human) recipient. To employ the cells in such a manner, one must prevent their rejection in a graft-versus-host response without compromising CAR-dependent effector functions.

In embodiments of this invention, T-cell receptor (TCR) a disruption from chimeric antigen receptor (CAR)-expressing T cells (CAR-T cells) to establish “universal” T cell-based immunotherapy is provided. Redirecting T-cell specificity to desired antigen can be achieved through CARs. However, ex vivo generation of CAR-T cells from patient is limited by time and expense. Moreover, T cells derived from patients are sometimes functionally flawed because of the multiple rounds of lymphotoxic (lymphodepleting) chemotherapy. To this end, embodiments of the present invention concern the generation of CAR-T cells from immortalized T cells that can serve as “off-the-shelf reagents. In other words, engineered immortalized T cells can be pre-prepared and then infused into multiple recipients. This will facilitate “centralized” manufacturing of the universal T cells and subsequent pre-positioning of the T cells at regional facilities for infusion on demand, enable clinical trials to be undertaken that are powered for efficacy, and facilitate combination therapies in which the universal T cells can be administered with other biologies and therapeutics. To achieve this, one can eliminate endogenous TCR and B2M expression, which causes unwanted allogeneic immune reactions. Such steps can occur by any suitable manner, including by introducing a Cas9/CRISPR complex, for example, targeting TCR α constant region or β constant region. Embodiments of the invention are unique as they combine (i) redirecting the specificity of immortalized T cells by introducing a CAR and (ii) eliminating expression of endogenous TCR and B2M to generate a desired T-cell product. In certain embodiments, the introduction of CAR and elimination of TCR/B2M are accomplished by electroporation to stably express CAR and desired transient transfection of in vitro-transcribed mRNA. In embodiments of the invention, infusing specific engineered immortalized CAR-T cells are pre-prepared and thawed to be infused on demand as an off-the-shelf reagent.

The inventors demonstrate that Cas9/CRISPR complexes targeting either the endogenous TCRs or B2M in T cells resulted in the desired loss of TCR expression. As expected, these modified T cells did not respond to TCR stimulation in a mixed lymphocyte reaction assay, but maintained their CAR mediated re-directed specificity for the exemplary antigen, BCMA.

In certain embodiments of the invention, immortalized T-cells are genetically modified ex vivo to express a chimeric antigen receptor (CAR) to redirect specificity to a tumor associated antigen (TAA) thereby conferring anti-tumor activity in vivo. T-cells expressing a BCMA-specific CAR recognize B-cell malignancies in multiple recipients independent of MHC because the specificity domains are cloned from anti-BCMA FN3 domains. The present invention encompasses a major step towards eliminating the need to generate patient-specific T cells by generating “universal” engineered immortalized TAA-specific T cells that might be administered to multiple recipients. This was achieved by genetically editing specific CAR T cells to eliminate expression of the endogenous TCRs and B2M to prevent a graft-versus-host response without compromising CAR-dependent effector functions. Genetically modified T cells were generated by permanently deleting TCRs and B2M with designer Cas9/CRISPR complexes followed by stably introducing the specific CAR of interest. The inventors show that these engineered T cells display the expected property of having redirected specificity for BCMA without responding to TCR stimulation. These engineered immortalized CAR-T cells may be used as off-the-shelf therapy for investigational treatment of many types of cancers.

In particular, to test the feasibility of using engineered immortalized CAR-T cells the inventors modified the culturing process for generating CAR-T cells to include the editing of the genome of the immortalized T cells to irreversibly eliminate expression of TCRs and B2M. To knockout the TCR and B2M loci the inventors developed Cas9/CRISPR complexes, comprised of DNA-binding domains fused to the DNA cleavage domain from the Cas9 endonuclease, targeting genomic sequences in the constant regions of the endogenous TCRs and B2M, Cas9/CRISPR mediate genome editing by catalyzing the formation of a DNA double strand break (DSB) in the genome. Targeting a DSB to a predetermined site within the coding sequence of a gene has been previously shown to lead to permanent loss of functional target gene expression via repair by non-homologous end joining (NHEJ), an error-prone cellular repair pathway that results in the insertion or deletion of nucleotides at the cleaved site (Santiago Y, Chan E, Liu P Q, Orlando S, Zhang L, Umov F D, Holmes M C, Guschin D, Waite A, Miller J C, Rebar E J, Gregory P D, Klug A, Collingwood T N (2008) Targeted gene knockout in mammalian cells by using engineered zinc-finger nucleases. Proc Natl Acad Sci USA. 105:5809-5814; Perez E E, Wang J, Miller J C, Jouvenot Y, Kim K A, Liu O, Wang N, Lee G, Bartsevich V V, Lee Y L, Guschin D Y, Rupniewski I, Waite A J, Carpenito C, Carroll R G, Orange J S, Umov F D, Rebar E J, Ando D, Gregory P D, Riley J L, Holmes M C, June C H (2008) Establishment of HIV-1 resistance in CD4+ T cells by genome editing using zincfinger nucleases. Nat Biotechnol 26:808-816)

### Chimeric Antigen Receptors

As used herein, the term “antigen” is a molecule capable of being bound by an antibody or T-cell receptor. An antigen is additionally capable of inducing a humoral immune response and/or cellular immune response leading to the production of B and/or T lymphocytes.

The present invention involves nucleic acids, including nucleic acids encoding an antigen-specific chimeric antigen receptor (CAR), including a CAR that has been humanized to reduce immunogenicity (hCAR), polypeptide comprising an intracellular signaling domain, a transmembrane domain, and an extracellular domain comprising one or more signaling motifs. In certain embodiments, the CAR may recognize an epitope comprised of the shared space between one or more antigens. In certain embodiments, the binding region can comprise complementary determining regions of a monoclonal antibody, variable regions of a monoclonal antibody, and/or antigen binding fragment thereof. A complementarity determining region (CDR) is a short amino acid sequence found in the variable domains of antigen receptor (e.g., immunoglobulin and T-cell receptor) proteins that complements an antigen and therefore provides the receptor with its specificity for that particular antigen. Each polypeptide chain of an antigen receptor contains three CDRs (CDR1, CDR2, and CDR3). Since the antigen receptors are typically composed of two polypeptide chains, there are six CDRs for each antigen receptor that can come into contact with the antigen-each heavy and light chain contains three CDRs. Because most sequence variation associated with immunoglobulins and T-cell receptors are found in the CDRs, these regions are sometimes referred to as hypervariable domains. Among these, CDR3 shows the greatest variability as it is encoded by a recombination of the VJ (VDJ in the case of heavy chain and TCR ac chain) regions. It is contemplated that the human CAR nucleic acids are human genes to enhance cellular immunotherapy for human patients.

In other embodiments, that specificity is derived from a non-naturally occurring FN3 domain designed from a consensus sequence of fifteen FN3 domains from human tenascin-C known as Tencon (Jacobs et al., Protein Engineering, Design, and Selection, 25:107-117, 2012; US2010/0216708). The crystal structure of Tencon shows six surface-exposed loops that connect seven beta-strands as is characteristic to the FN3 domains, the beta-strands referred to as A, B, C, D, E, F, and G, and the loops referred to as AB, BC, CD, DE, EF, and FG loops (Bork and Doolittle, PNAS USA 89:8990-8992, 1992; U.S. Pat. No. 6,673,901). These loops, or selected residues within each loop, can be randomized in order to construct libraries of FN3 domains that can be used to select novel molecules that bind the antigen of interest. Libraries designed based on the Tencon sequence (SEQ ID NO: 1) can thus have randomized sequence in one or more of the loops or strands. For example, libraries based on Tencon can have randomized sequence in one or more of the AB loop, BC loop, CD loop, DE, EF loop and FG loop. For example, the Tencon BC loop is 7 amino acids long, thus 1, 2, 3, 4, 5, 6 or 7 amino acids can be randomized in a library based on Tencon sequence, diversified at the BC loop. The Tencon CD loop is 6 amino acids long, thus 1, 2, 3, 4, 5 or 6 amino acids can be randomized in a library based on Tencon sequence, diversified at the CD loop. The Tencon EF loop is 5 amino acids long, thus 1, 2, 3, 4 or 5 amino acids can be randomized in a library based on Tencon sequence, diversified at the EF loop. The Tencon FG loop is 7 amino acids long, thus 1, 2, 3, 4, 5, 6 or 7 amino acids can be randomized in a library based on Tencon sequence, diversified at the FG loop. Further diversity at loops in the Tencon libraries can be achieved by insertion and/or deletions of residues at loops. For example, the BC, CD, EF and/or FG loops can be extended by 1-22 amino acids or decreased by 1-3 amino acids. The FG loop in Tencon is 7 amino acids long, whereas the corresponding loop in antibody heavy chains ranges from 4-28 residues. To provide maximum diversity, the FG loop can be diversified in sequence as well as in length to correspond to the antibody CDR3 length range of 4-28 residues. For example, the FG loop can be further diversified in length by extending the loop by an additional 1, 2, 3, 4 or 5 amino acids. Libraries designed based on the Tencon sequence can also have randomized alternative surfaces that form on a side of the FN3 domain and comprise two or more beta strands, and at least one loop. One such alternative surface is formed by amino acids in the C and the F beta-strands and the CD and the FG loops (a C-CD-F-FG surface). A library design based on Tencon alternative C-CD-F-FG surface is described in US2013/0226834. Libraries designed based on the Tencon sequence also includes libraries designed based on Tencon variants, such as Tencon variants having substitutions at residues positions 11, 17, 46 and/or 86, and which variants display improve thermal stability. Exemplary Tencon variants are described in US2011/0274623 and include Tencon27 (SEQ ID NO: 2) having substitutions E11R, L17A, N46V and E861 when compared to Tencon. Tencon libraries and other FN3 sequence-based libraries can be randomized at chosen residue positions using a random or defined set of amino acids. For example, variants in the library having random substitutions can be generated using NNK codons, which encode all 20 naturally occurring amino acids. In other diversification schemes, DVK codons can be used to encode amino acids Ala, Trp, Tyr, Lys, Thr, Asn, Lys, Ser, Arg, Asp, Glu, Gly, and Cys. Alternatively, NNS codons can be used to give rise to all 20 amino acid residues while simultaneously reducing the frequency of stop codons. Libraries of FN3 domains with biased amino acid distribution at positions to be diversified can be synthesized, for example, using Slonomics® technology (http:_//www_sloning_com). This technology uses a library of pre-made double stranded triplets that act as universal building blocks sufficient for thousands of gene synthesis processes. The triplet library represents all possible sequence combinations necessary to build any desired DNA molecule. The codon designations are according to the well known IUB code.

In a specific embodiment, the invention includes a full-length CAR cDNA or coding region. The antigen binding regions or domain can comprise a fragment of the VH and VL chains of a single-chain variable fragment (scFv) derived from a particular human monoclonal antibody. The antigen binding regions or domain can also comprise an FN3 domain.

The intracellular signaling domain of the chimeric receptor of the invention is responsible for activation of at least one of the normal effector functions of the immune cell in which the chimeric receptor has been placed. The term “effector function” refers to a specialized function of a differentiated cell. Effector function of a T cell, for example, may be cytolytic activity or helper activity including the secretion of cytokines. Effector function in a memory or memory-type T cell includes antigen-dependent proliferation. Thus the term “intracellular signaling domain” refers to the portion of a protein that transduces the effector function signal and directs the cell to perform a specialized function. While usually the entire intracellular signaling domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term intracellular signaling domain is thus meant to include any truncated portion of the intracellular signaling domain sufficient to transduce the effector function signal. Examples include the zeta chain of the T-cell receptor or any of its homo logs (e.g., eta, delta, gamma, or epsilon), MB1 chain, B29, FcyRUT, FcyR, and combinations of signaling molecules, such as OO3ζ and CD2.8, 4-1BB, OX40, and combination thereof, as well as other similar molecules and fragments. Intracellular signaling portions of other members of the families of activating proteins can be used, such as FcyRIII and FcsRL See Gross. G., et al., “Endowing T Cells With Antibody Specificity Using Chimeric T Cell Receptors,” FASEB J., vol 6, 1992, pp. 3370-3378, Stancovski, I., et al, “Targeting of T Lymphocytes to Neu/HER2-Expressing Cells Using Chimeric Single Chain Fv Receptors,” J. Immunol., vol. 151, 1993, pp. 6377-6382, Moritz, D., et al., “Cytotoxic T Lymphocytes with a Grafted Recognition Specificity for ERBB2-Expressing Tumor Cells,” PNAS USA, vol. 91, 1994, pp. 4318-4322,

Hwu, P., Yang, J. C., Cowherd, R., Treisman, J., Shafer, G. E., Eshhar, Z. and Rosenberg, S. A., In vivo activity of T cells redirected with chimeric antibody/T-cell receptor genes. Cancer Res., 55, 3369-3373 (1995),
Weijtens, M. E., Willemsen, R. A., Valeno, D., Stam, K. and Bolhuis, R. L., Single chain Ig/gamma gene-redirected human T lymphocytes produce cytokines, specifically lyse tumor cells, and recycle lytic capacity. J Immunol., 157, 836-843 (1996), and Hekele, A., Dall, P., Moritz, D., Wels, W., Groner, B., Herrlich, P. and Ponta, H., Growth retardation of tumors by adoptive transfer of cytotoxic T lymphocytes reprogrammed by CD44v6-specific scFv.zeta-chimera. Int. J. Cancer, 68, 232-238 (1996) for disclosures of cTCR's using these alternative transmembrane and intracellular domains. In a preferred embodiment, the human CD3 ζ intracellular domain was taken for activation.

The antigen-specific extracellular domain and the intracellular signaling-domain may be linked by a transmembrane domain, such as the human IgG₄Fc hinge and Fc regions, human CD4 transmembrane domain, the human CD28 transmembrane domain, the transmembrane human CD3 domain, or a cysteine mutated human€O3ζ domain, or other transmembrane domains from other human transmembrane signaling proteins, such as CD 16 and CD8 and erythropoietin receptor.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, OX-40 (CD 134), DAP 10, and 4-IBB (CD137). In addition to a primary signal initiated by CD3, an additional signal provided by a human costimulatory receptor inserted in a human CAR is important for full activation of T cells and could help improve in vivo persistence and the therapeutic success of the adoptive immunotherapy. In particular embodiments, the invention concerns isolated nucleic acid segments and expression cassettes incorporating DNA sequences that encode the CAR. Vectors of the present invention are designed, primarily, to deliver desired genes to immune cells, preferably T cells under the control of regulated eukaryotic promoters, for example, MNDU3 promoter or EFlapha promoter, or Ubiquitin promoter. Also, the vectors may contain a selectable marker if for no other reason, to facilitate their manipulation in vitro.

Chimeric antigen receptor molecules are recombinant and are distinguished by their ability to both bind antigen and transduce activation signals via immunoreceptor activation motifs (ITAM's) present in their cytoplasmic tails. Receptor constructs utilizing an antigen-binding moiety (for example, generated from single chain antibodies (scFv)) afford the additional advantage of being “universal” in that they bind native antigen on the target cell surface in an HLA-independent fashion. For example, several laboratories have reported on scFv constructs fused to sequences coding for the intracellular portion of the CD3 complex's zeta chain (ζ), the Fc receptor gamma chain, and sky tyrosine kinase (Eshhar et al, Proc, Natl Acad. Sci. U.S.A., 90:720, 1993). Re-directed T cell effector mechanisms including tumor recognition and lysis by CTL have been documented in several murine and human antigen-scFv: ζ systems (Altenschmidt et al, J. Mol Med, 75:259, 1997).

To date non-human antigen binding regions are typically used in constructing a chimeric antigen receptor. A potential problem with using non-human antigen binding regions, such as murine monoclonal antibodies, is the lack of human effector functionality and inability to penetrate into tumor masses. In other words, such antibodies may be unable to mediate complement-dependent lysis or lyse human target cells through antibody-dependent cellular toxicity or Fc-receptor mediated phagocytosis to destroy cells expressing CAR. Furthermore, non-human monoclonal antibodies can be recognized by the human host as a foreign protein, and therefore, repeated injections of such foreign antibodies can lead to the induction of immune responses leading to harmful hypersensitivity reactions. For murine-based monoclonal antibodies, this is often referred to as a Human Anti-Mouse Antibody (HAMA) response. Therefore, the use of human antibodies is more preferred because they do not elicit as strong a HAMA response as murine antibodies. Similarly, the use of human sequences in the CAR can avoid immune-mediated recognition and therefore elimination by endogenous T cells that reside in the recipient and recognize processed antigen in the context of HLA. In some embodiments, the chimeric antigen receptor comprises: (a) an extracellular domain comprising an antigen binding region; (b) a transmembrane domain; and (c) an intracellular signaling domain.

In specific embodiments, intracellular receptor signaling domains in the CAR include those of the T cell antigen receptor complex, such as the zeta chain of CD3, also Fcgamma RIII costimulatory signaling domains, CD28, DAP 10, CD2, alone or in a series with CD3zeta, for example. In specific embodiments, the intracellular domain (which may be referred to as the cytoplasmic domain) comprises part or all of one or more of TCR zeta chain, CD28, OX40/CD134, 4-1BB/CD137, FcsRTy, ICOS/CD278, ILRB/CD122, IL-2RG/CD132, DAP molecule, CD27, DAP 10, DAP 12, and CD40. In some embodiments, one employs any part of the endogenous T cell receptor complex in the intracellular domain. One or multiple cytoplasmic domains may be employed, as so-called third generation CARs have at least two or three signaling domains fused together for additive or synergistic effect, for example. In certain embodiments of the chimeric antigen receptor, the antigen-specific portion of the receptor (which may be referred to as an extracellular domain comprising an antigen binding region) comprises a tumor associated antigen or a pathogen-specific antigen.

A tumor associated antigen may be of any kind so long as it is expressed on the cell surface of tumor cells. Exemplary embodiments of tumor associated antigens include BCMA, CD19, CD20, carcinoembryonic antigen, alphafetoprotein, CA-125, MUC-1, epithelial tumor antigen, melanoma-associated antigen, mutated p53, mutated ras, and so forth.

In certain embodiments, intracellular tumor associated antigens may be targeted, such as HA-1, WTI, or p53. This can be achieved by a CAR expressed on a universal T cell that recognizes the processed peptide described from the intracellular tumor associated antigen in the context of HLA. In addition, the universal T cell may be genetically modified to express a T-cell receptor pairing that recognizes the intracellular processed tumor associated antigen in the context of HLA.

The pathogen may be of any kind, but in specific embodiments the pathogen is a fungus, bacteria, or virus, for example. Exemplary viral pathogens include those of the families of Adenoviridae, Epstein-Barr virus (EBV), Cytomegalovirus (CMV), Respiratory Syncytial Virus (RSV), JC virus, BK virus, HSV, HHV family of viruses, Picomaviridae, Herpesviridae, Hepadnaviridae, Flaviviridae, Retroviridae, Orthomyxoviridae, Parainyxoviridae, Papovaviridae, Polyomavirus, Rhabdoviridae, and Togavkidae. Exemplary pathogenic viruses cause smallpox, influenza, mumps, measles, chickenpox, ebola, and rubella. Exemplary pathogenic fungi include Candida, Aspergillus, Cryplococcus, Histoplasma, Pneumocystis, and Stachybotrys. Exemplary pathogenic bacteria include Streptococcus, Pseudomonas, Shigella, Campylobacter, Staphylococcus, Helicobacter, E, coli, Rickettsia, Bacillus, Bordetella, Chlamydia, Spirochetes, and Salmonella. In one embodiment, the pathogen receptor Dectin-1 can be used to generate a CAR that recognizes the carbohydrate structure on the cell wall of fungi. T cells genetically modified to express the CAR based on the specificity of Dectin-1 can recognize Aspergillus and target hyphal growth. In another embodiment, CARs can be made based on an antibody recognizing viral determinants (e.g., the glycoproteins from CMV and Ebola) to interrupt viral infections and pathology. In some embodiments, the pathogenic antigen is an Aspergillus carbohydrate antigen for which the extracellular domain in the CAR recognizes patterns of carbohydrates of the fungal cell wall.

A chimeric immunoreceptor according to the present invention can be produced by any means known in the art, though preferably it is produced using recombinant DMA techniques. A nucleic acid sequence encoding the several regions of the chimeric receptor can be prepared and assembled into a complete coding sequence by standard techniques of molecular cloning (genomic library screening, PCR, primer-assisted ligation, scFv libraries from yeast and bacteria, site-directed mutagenesis, etc.). The resulting coding region can be inserted into an expression vector and used to transform a suitable expression host immortalized T cell line. As used herein, a “nucleic acid construct” or “nucleic acid sequence” or “polynucleotide” is intended to mean a DNA molecule that can be transformed or introduced into a T cell and be transcribed and translated to produce a product (e.g., a chimeric receptor).

In an exemplary nucleic acid construct (polynucleotide) employed in the present invention, the promoter is operably linked to the nucleic acid sequence encoding the chimeric receptor of the present invention, i.e., they are positioned so as to promote transcription of the messenger RNA from the DNA encoding the chimeric receptor. The promoter can be of genomic origin or synthetically generated. A variety of promoters for use in T cells are well-known in the art (e.g., the CD4 promoter disclosed by Marodon et al., Blood, 101:3416-3423, 2003). The promoter can be constitutive or inducible, where induction is associated with the specific cell type or a specific level of maturation, for example. Alternatively, a number of well-known viral promoters are also suitable. Promoters of interest include the β-actin promoter, SV40 early and late promoters, immunoglobulin promoter, human cytomegalovirus promoter, retrovirus promoter, and the Friend spleen focus-forming virus promoter. The promoters may or may not be associated with enhancers, wherein the enhancers may be naturally associated with the particular promoter or associated with a different promoter. The sequence of the open reading frame encoding the chimeric receptor can be obtained from a. genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA or provide T cell-specific expression (Barthel and Goidfeld, Immunol, 171:3612-3619, 2003). Also, it may be further advantageous to use endogenous or exogenous non-coding regions to stabilize the mRNA.

For expression of a chimeric receptor of the present invention, the naturally occurring or endogenous transcriptional initiation region of the nucleic acid sequence encoding N-termini components of the chimeric receptor can be used to generate the chimeric receptor in the target host. Alternatively, an exogenous transcriptional initiation region can be used that allows for constitutive or inducible expression, wherein expression can be controlled depending upon the target host, the level of expression desired, the nature of the target host, and the like.

Likewise, a signal sequence directing the chimeric receptor to the surface membrane can be the endogenous signal sequence of N-terminal component of the chimeric receptor. Optionally, in some instances, it may be desirable to exchange this sequence for a different signal sequence. However, the signal sequence selected should be compatible with the secretory pathway of T cells so that the chimeric receptor is presented on the surface of the T cell. Similarly, a termination region may be provided by the naturally occurring or endogenous transcriptional termination region of the nucleic acid sequence encoding the C-terminal component of the chimeric receptor. Alternatively, the termination region may be derived from a different source. For the most part, the source of the termination region is generally not considered to be critical to the expression of a recombinant protein and a wide variety of termination regions can be employed without adversely affecting expression. As will be appreciated by one of skill in the art that, in some instances, a few amino acids at the ends of the antigen binding domain in the CAR can be deleted, usually not more than 10, more usually not more than 5 residues, for example. Also, it may be desirable to introduce a small number of amino acids at the borders, usually not more than 10, more usually not more than 5 residues. The deletion or insertion of amino acids may be as a result of the needs of the construction, providing for convenient restriction sites, ease of manipulation, improvement in levels of expression, or the like. In addition, the substitute of one or more amino acids with a different amino acid can occur for similar reasons, usually not substituting more than about five amino acids in any one domain.

The chimeric construct that encodes the chimeric receptor according to the invention can be prepared in conventional ways. Because, for the most part, natural sequences may be employed, the natural genes may be isolated and manipulated, as appropriate, so as to allow for the proper joining of the various components. Thus, the nucleic acid sequences encoding for the N-terminal and C-terminal proteins of the chimeric receptor can be isolated by employing the polymerase chain reaction (PCR), using appropriate primers that result in deletion of the undesired portions of the gene. Alternatively, restriction digests of cloned genes can be used to generate the chimeric construct. In either case, the sequences can be selected to provide for restriction sites that are blunt-ended, or have complementary overlaps.

The various manipulations for preparing the chimeric construct can be carried out in vitro, and in particular embodiments, the chimeric construct is introduced into vectors for cloning and expression in an appropriate host using standard transformation or transfection methods. Thus, after each manipulation, the resulting construct from joining of the DNA sequences is cloned, the vector isolated, and the sequence screened to ensure that the sequence encodes the desired chimeric receptor. The sequence can be screened by restriction analysis, sequencing, or the like. The chimeric constructs of the present invention find application in subjects having or suspected of having cancer by reducing the size of a tumor or preventing the growth or re-growth of a tumor in these subjects. Accordingly, the present invention further relates to a method for reducing growth or preventing tumor formation in a subject by introducing a chimeric construct of the present invention into an engineered immortalized T cell and introducing into the subject the engineered immortalized CAR-T cell, thereby effecting anti-tumor responses to reduce or eliminate tumors in the subject. Suitable immortalized T cells that can be used include cytotoxic lymphocytes (CTL) or any immortalized cell having a T cell receptor in need of disruption.

It is contemplated that the chimeric construct can be introduced into the immortalized T cells as naked DNA or in a suitable vector. Methods of stably transfecting T cells by electroporation using naked DNA are known in the art. See, e.g., U.S. Pat. No. 6,410,319. Naked DNA generally refers to the DNA encoding a chimeric receptor of the present invention contained in a plasmid expression vector in proper orientation for expression. Advantageously, the use of naked DNA reduces the time required to produce immortalized T cells expressing the chimeric receptor of the present invention.

Alternatively, a viral vector (e.g., a retroviral vector, adenoviral vector, adeno-associated viral vector, or lentiviral vector) can be used to introduce the chimeric construct into immortalized T cells. Suitable vectors for use in accordance with the method of the present invention are non-replicating in the immortalized T cells. A large number of vectors are known that are based on viruses, where the copy number of the virus maintained in the cell is low-enough to maintain the viability of the cell. Illustrative vectors include the pFB-neo vectors (STRATAGENE®), as well as vectors based on HIV, SV40, EBV, HSV, AAV or BPV.

Once it is established that the transfected or transduced immortalized T cell is capable of expressing the chimeric receptor as a surface membrane protein with the desired regulation and at a desired level, it can be determined whether the chimeric receptor is functional in the host cell to provide for the desired signal induction. Subsequently, the transduced immortalized T cells are reintroduced or administered to the subject to activate anti-tumor responses in the subject. To facilitate administration, the transduced T cells according to the invention can be made into a pharmaceutical composition or made into an implant appropriate for administration in vivo, with appropriate carriers or diluents, which further can be pharmaceutically acceptable. The means of making such a composition or an implant have been described in the art (see, for instance. Remington's Pharmaceutical Sciences, 16th Ed., Mack, ed. (1980)). Where appropriate, the transduced immortalized T cells can be formulated into a preparation in semisolid or liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Desirably, however, a pharmaceutically acceptable form is employed that does not ineffectuate the cells expressing the chimeric receptor. Thus, desirably the transduced immortalized T cells can be made into a pharmaceutical composition containing a balanced salt solution, preferably Hanks' balanced salt solution, or normal saline.

### Exemplary BCMA-Specific Chimeric T-Cell Receptor (or Chimeric Antigen Receptor, CAR)

A potential target for MM therapies is B cell maturation antigen (BCMA), a member of the tumor necrosis factor receptor family that is predominantly expressed on mature B cells (Coquery and Erickson, Crit Rev Immunol. 2012; 32(4):287-305). BCMA delivers pro-survival signals upon binding to its ligands, B cell activator of the TNF family (BAFF) and a proliferation inducing ligand (APRIL). BCMA triggers antigen presentation in B cells that is dependent on NF-κB and JNK signaling. In healthy individuals, BCMA plays a role in mediating the survival of plasma cells that maintain long-term humoral immunity, but its expression has also been linked to a number of cancers, autoimmune disorders, and infectious diseases. For example, BCMA RNA has been detected universally in MM cells and in other lymphomas, and BCMA protein has been detected on the surface of plasma cells from MM patients (Novak et al., Blood. 2004 Jan. 15; 103(2):689-94; Neri et al., Clin Cancer Res. 2007 Oct. 1; 13(19):5903-9; Bellucci et al., Blood. 2005 May 15; 105(10):3945-50; Moreaux et al., Blood. 2004 Apr. 15; 103(8):3148-57).

In one aspect, compositions of the invention include a BCMA-targeting CAR comprising a BCMA-specific FN3 domain.

In one aspect, the invention relates to a CAR comprising:
a. an extracellular domain having an FN3 domain that specifically binds to a BCMA;
b. a transmembrane domain; and
c. an intracellular signaling domain.

In some embodiments, in a nascent CAR, the extracellular domain is preceded by a signal peptide at the N-terminus. Any suitable signal peptide can be used in the invention. The signal peptide can be derived from a natural, synthetic, semi-synthetic or recombinant source. According to one embodiment, the signal peptide is a human CD8 signal peptide, a human CD3 delta signal peptide, a human CD3 epsilon signal peptide, a human GMCSFR signal peptide, a human 4-1BB signal peptide, or a derivative thereof. According to particular embodiments, the signal peptide has an amino acid sequence at least 90% identical to SEQ ID NO: 3, preferably the amino acid sequence of SEQ ID NO: 3. According to other particular embodiments, the signal peptide has an amino acid sequence at least 90% identical to one of SEQ ID NOs: 46-49, preferably the amino acid sequence of one of SEQ ID NOs: 50-53. The signal peptide can be cleaved by a signal peptidase during or after completion of translocation to generate a mature CAR free of the signal peptide.

According to embodiments of the invention, the extracellular domain of a CAR comprises a BCMA-specific FN3 domain. Any BCMA-specific FN3 domain according to embodiments of the invention, including but not limited to amino acid sequences, according to SEQ ID NOs 8-44, can be used in the extracellular domain of the CAR.

According to embodiments of the invention, a CAR can further comprise a hinge region connecting the extracellular domain and the transmembrane domain. The hinge region functions to move the extracellular domain away from the surface of the engineered immune cell to enable proper cell/cell contact, binding to the target or antigen and activation (Patel et al., Gene Therapy, 1999; 6: 412-419). Any suitable hinge region can be used in a CAR of the invention. It can be derived from a natural, synthetic, semi-synthetic or recombinant source. According to some embodiments, the hinge region of the CAR is a 6×GS peptide (SEQ ID NO: 66), or a fragment thereof, or a hinge region from a CD8 protein, or a derivative thereof. In particular embodiments, the hinge region has an amino acid sequence at least 90% identical to SEQ ID NO: 4, preferably the amino acid sequence of SEQ ID NO: 4.

Any suitable transmembrane domain can be used in a CAR of the invention. The transmembrane domain can be derived from a natural, synthetic, semi-synthetic or recombinant source. According to some embodiments, the transmembrane domain is a transmembrane domain from molecules such as CD8, CD28, CD4, CD2, GMCSFR and the like. In particular embodiments, the transmembrane domain has an amino acid sequence at least 90% identical to SEQ ID NO: 5, preferably the amino acid sequence of SEQ ID NO: 5. In other embodiments, the transmembrane domain has an amino acid sequence at least 90% identical to one of SEQ ID NOs: 50-53, preferably the amino acid sequence of one of SEQ ID NOs: 50-53.

Any suitable intracellular signaling domain can be used in a CAR of the invention. In particular embodiments, the entire intracellular signaling domain is used. In other particular embodiments, a truncated portion of the signaling domain that transduces the effector signal is used. According to embodiments of the invention, the intracellular signaling domain generates a signal that promotes an immune effector function of the CAR-containing cell, e.g. a CAR-T cell, including, but not limited to, proliferation, activation, and/or differentiation. In particular embodiments, the signal promotes, e.g., cytolytic activity, helper activity, and/or cytokine secretion of the CAR-T cell.

According to some embodiments, the intracellular signaling domain comprises a functional signaling domain derived from CD3 zeta, TCR zeta, FcR gamma, FcR beta, CD3 gamma, CD3 delta, CD3 epsilon, CD16, CD22, CD27, CD28, CD30, CD79a, CD79b, CD134 (also known as TNFRSF4 or OX-40), 4-1BB (CD137), CD278 (also known as ICOS), FcεRI, DAP10, DAP12, ITAM domains or CD66d, and the like.

According to particular embodiments, the intracellular signaling domain comprises a primary signaling domain and one or more co-stimulatory signaling domains.

In one embodiment, the intracellular signaling domain comprises a primary intracellular signaling domain having a functional signaling domain derived from human CD3zeta. In particular embodiments, the primary intracellular signaling domain has an amino acid sequence at least 90% identical to SEQ ID NO: 7, preferably the amino acid sequence of SEQ ID NO: 7.

According to some embodiments, the intracellular signaling domain further comprises the co-stimulatory intracellular signaling domain derived from human 4-1BB. In particular embodiments, the co-stimulatory intracellular signaling domain has an amino acid sequence at least 90% identical to SEQ ID NO: 6, preferably the amino acid sequence of SEQ ID NO: 6.

In one embodiment, the intracellular signaling domain has an amino acid sequence at least 90% identical to SEQ ID NO: 45, preferably the amino acid sequence of SEQ ID NO: 45.

In particular embodiments, a CAR has the structure comprising, from the N-terminus to the C-terminus, a BCMA-specific FN3 domain (Centyrin), a human CD8 hinge region, a human CD8 transmembrane region, a human 4-1BB intracellular domain, and a human CD3 zeta intracellular domain. The nascent CAR further comprises a human CD8 signal peptide, which is subsequently cleaved in the mature CAR.

In one embodiment, a CAR of the invention is associated with a host cell expressing the CAR.

In another embodiment, a CAR of the invention is present in an engineered immortalized T cell.

In yet another embodiment, a CAR of the invention is purified or isolated from other components of the host cell expressing the CAR.

### Exemplary FN3 Domain-Targeting Chimeric T-Cell Receptor (or Chimeric Antigen Receptor, CAR)

In other general aspects, the invention relates to an FN3 domain-targeting CAR comprising an FN3 domain-specific scFv.

In one aspect, the invention relates to a CAR comprising:
a. an extracellular domain having an scFv that specifically binds to a non-randomized region of an FN3 domain;
b. a transmembrane domain; and
c. an intracellular signaling domain.

CARs comprising an FN3 domain-specific scFv can be used to control T-cell mediated killing using targeted FN3 domains that can be pre-loaded onto engineered cells or dosed and controlled to prevent toxicity. Also, non-targeting FN3 domains can be conjugated with ligands to engage other cell types in a ligand/receptor specific manner, or to achieve selectivity to engage multiple ligands at the same time.

In some embodiments, in a nascent CAR, the extracellular domain is preceded by a signal peptide at the N-terminus. Any suitable signal peptide can be used in the invention. The signal peptide can be derived from a natural, synthetic, semi-synthetic or recombinant source.

According to embodiments of the invention, the extracellular domain of a CAR comprises an scFv that specifically binds to a non-randomized region of an FN3 domain. Any scFv that specifically binds to an FN3 domain according to embodiments of the invention, including but not limited to amino acid sequences, according to SEQ ID NOs: 54 and 55, can be used in the extracellular domain of the CAR.

In some embodiments, in a nascent CAR, the extracellular domain is preceded by a signal peptide at the N-terminus. Any suitable signal peptide can be used in the invention. The signal peptide can be derived from a natural, synthetic, semi-synthetic or recombinant source. According to one embodiment, the signal peptide is a human CD8 signal peptide, a human CD3 delta signal peptide, a human CD3 epsilon signal peptide, a human GMCSFR signal peptide, a human 4-1BB signal peptide, or a derivative thereof. According to particular embodiments, the signal peptide has an amino acid sequence at least 90% identical to SEQ ID NO: 3, preferably the amino acid sequence of SEQ ID NO: 3. According to other particular embodiments, the signal peptide has an amino acid sequence at least 90% identical to one of SEQ ID NOs: 46-49, preferably the amino acid sequence of one of SEQ ID NOs: 50-53. The signal peptide can be cleaved by a signal peptidase during or after completion of translocation to generate a mature CAR free of the signal peptide.

Any suitable transmembrane domain can be used in a CAR of the invention. The transmembrane domain can be derived from a natural, synthetic, semi-synthetic or recombinant source. According to some embodiments, the transmembrane domain is a transmembrane domain from molecules such as CD8, CD28, CD4, CD2, GMCSFR and the like. In particular embodiments, the transmembrane domain has an amino acid sequence at least 90% identical to SEQ ID NO: 5, preferably the amino acid sequence of SEQ ID NO: 5. In other embodiments, the transmembrane domain has an amino acid sequence at least 90% identical to one of SEQ ID NOs: 50-53, preferably the amino acid sequence of one of SEQ ID NOs: 50-53.

Any suitable intracellular signaling domain can be used in a CAR of the invention. In particular embodiments, the entire intracellular signaling domain is used. In other particular embodiments, a truncated portion of the signaling domain that transduces the effector signal is used. According to embodiments of the invention, the intracellular signaling domain generates a signal that promotes an immune effector function of the CAR-containing cell, e.g. a CAR-T cell, including, but not limited to, proliferation, activation, and/or differentiation. In particular embodiments, the signal promotes, e.g., cytolytic activity, helper activity, and/or cytokine secretion of the CAR-T cell. In other embodiments, no intracellular signaling domain is used in a CAR of the invention and the CAR comprising an scFv that specifically binds to an FN3 domain of the invention is used along with an FN3 domain for targeting the effector cell to target cells.

According to some embodiments, the intracellular signaling domain comprises a functional signaling domain derived from CD3 zeta, TCR zeta, FcR gamma, FcR beta, CD3 gamma, CD3 delta, CD3 epsilon, CD16, CD22, CD27, CD28, CD30, CD79a, CD79b, CD134 (also known as TNFRSF4 or OX-40), 4-1BB (CD137), CD278 (also known as ICOS), FcεRI, DAP10, DAP12, ITAM domains or CD66d, and the like.

According to particular embodiments, the intracellular signaling domain comprises a primary signaling domain and one or more co-stimulatory signaling domains.

In one embodiment, the intracellular signaling domain comprises a primary intracellular signaling domain having a functional signaling domain derived from human CD3zeta. In particular embodiments, the primary intracellular signaling domain has an amino acid sequence at least 90% identical to SEQ ID NO: 7, preferably the amino acid sequence of SEQ ID NO: 7.

According to some embodiments, the intracellular signaling domain further comprises the co-stimulatory intracellular signaling domain derived from human 4-1BB. In particular embodiments, the co-stimulatory intracellular signaling domain has an amino acid sequence at least 90% identical to SEQ ID NO: 6, preferably the amino acid sequence of SEQ ID NO: 6.

In one embodiment, a CAR of the invention is associated with a host cell expressing the CAR.

In another embodiment, a CAR of the invention is present in an isolated cell membrane of the host cell expressing the CAR.

In yet another embodiment, a CAR of the invention is purified or isolated from other components of the host cell expressing the CAR.

### Exemplary Endonucleases for Disrupting TCRs and B2M

As a result of the present invention, engineered immortalized T-cells can be obtained having improved characteristics. In particular, the present invention provides an engineered, preferably immortalized, T-cell, which is characterized in that the expression of TCRs and B2M is inhibited.

According to certain embodiments, the engineered immortalized T-cell expresses an endonuclease able to selectively inactivate by DNA cleavage the gene of interest such as a gene encoding a TCR or B2M. The term “endonuclease” refers to a wild type or variant enzyme capable of catalyzing the hydrolysis (cleavage) of bonds between nucleic acids within a DNA or RNA molecule, preferably a DNA molecule. Particularly, said endonuclease is highly specific, recognizing nucleic acid target sites ranging from 10 to 45 base pairs (bp) in length, usually ranging from 10 to 35 base pairs in length, more usually from 12 to 20 base pairs. The endonuclease according to the present invention recognizes at specific polynucleotide sequences, further referred to as “target sequence” and cleaves nucleic acid inside these target sequences or into sequences adjacent thereto, depending on the molecular structure of said endonuclease. The endonuclease can recognize and generate a single- or double-strand break at specific polynucleotides sequences.

In particular embodiments, said endonuclease is the Cas9/CRISPR complex. Cas9/CRISPR endonuclease constitutes a new generation of genome engineering tool where Cas9 associates with a RNA molecule. In this system, the RNA molecule nucleotide sequence determines the target specificity and activates the endonuclease (Gasiunas, G. et al. (2012). “Cas9-crRNA ribonucleoprotein complex mediates specific DNA cleavage for adaptive immunity in bacteria.” Proc Natl Acad Sci USA 109(39): E2579-86; Jinek, M., K. Chylinski, et al. (2012). “A programmable dual-RNA-guided DNA endonuclease in adaptive bacterial immunity.” Science 337(6096): 816-21: Cong, L., F. A. Ran, et al. (2013). “Multiplex genome engineering using CRISPR/Cas systems.” Science 339(6121): 819-23; Mali, P., L. Yang, et al. (2013). “RNA-guided human genome engineering via Cas9. ” Science 339(6121); 823-6). Cas9, also named Csnl is a large protein that participates in both crRNA biogenesis and in the destruction of invading DNA. Cas9 has been described in different bacterial species such as S. thermophiles, Listeria innocua (Gasiunas, Barrangou et al. 2012; Jinek, Chylinski et al. 2012) and S. Pyogenes (Deltcheva, E., K. Chylinski, et al. (2011). “CRISPR RNA maturation by trans-encoded small RNA and host factor RNase 11I.” Nature 471(7340): 602-7). The large Cas9 protein (>1200 amino acids) contains two predicted nuclease domains, namely HNH (McrA-like) nuclease domain that is located in the middle of the protein and a splitted RuvC-like nuclease domain (RNase H fold). Cas9 variants can be a Cas9 endonuclease that does not naturally exist in nature and that is obtained by protein engineering or by random mutagenesis. Cas9 variants according to the invention can for example be obtained by mutations i.e. deletions from, or insertions or substitutions of at least one residue in the amino acid sequence of a Si pyogenes Cas9 endonuclease (COG3513).

In other embodiments, said endonuclease can also be a homing endonuclease, also known under the name of meganuclease. Such homing endonucleases are well-known to the art (Stoddard, B. L. (2005). “Homing endonuclease structure and function.” Q Rev Biophys 38(1): 49-95). Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG (SEQ ID NO: 67) endonuclease, to a HNH endonuclease, or to a GIY-YIG endonuclease. Preferred homing endonuclease according to the present invention can be an I-Crel variant. A “variant” endonuclease, i.e. an endonuclease that does not naturally exist in nature and that is obtained by genetic engineering or by random mutagenesis can bind DNA sequences different from that recognized by wild-type endonucleases (see international application WO2006/097854).

In other embodiments, said rare-cutting endonuclease can be a “Zinc Finger Nucleases” (ZFNs), which are generally a fusion between the cleavage domain of the type IIS restriction enzyme, Fokl, and a DNA recognition domain containing 3 or more C2H2 zinc finger motifs. The heterodimerization at a particular position in the DNA of two individual ZFNs in precise orientation and spacing leads to a double-strand break (DSB) in the DNA. The use of such chimeric endonucleases have been extensively reported in the art as reviewed by Umov et al. (Genome editing with engineered zinc finger nucleases (2010) Nature reviews Genetics 11:636-646). Standard ZFNs fuse the cleavage domain to the C-terminus of each zinc finger domain. In order to allow the two cleavage domains to dimerize and cleave DNA. the two individual ZFNs bind opposite strands of DNA with their C-termini a certain distance apart. The most commonly used linker sequences between the zinc finger domain and the cleavage domain requires the 5′ edge of each binding site to be separated by 5 to 7 bp. The most straightforward method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Numerous selection methods have been used to generate zinc-finger arrays capable of targeting desired sequences. Initial selection efforts utilized phage display to select proteins that bound a given DNA target from a large pool of partially randomized zinc-finger arrays. More recent efforts have utilized yeast one-hybrid systems, bacterial one-hybrid and two-hybrid systems, and mammalian cells.

In other embodiments, said endonuclease is a “TALE-nuclease” or a “MBBBD-nuclease” resulting from the fusion of a DNA binding domain typically derived from Transcription Activator Like Effector proteins (TALE) or from a Modular Base-per-Base Binding domain (MBBBD), with a catalytic domain having endonuclease activity. Such catalytic domain usually comes from enzymes, such as for instance I-Tevl, CoIE7, NucA and Fok-I. TALE-nuclease can be formed under monomeric or dimeric forms depending of the selected catalytic domain (WO2012138927). Such engineered TALE-nucleases are commercially available under the trade name TALEN™ (Cellectis, 8 rue de la Croix Jarry, 75013 Paris, France). In general, the DNA binding domain is derived from a Transcription Activator like Effector (TALE), wherein sequence specificity is driven by a series of 33-35 amino acids repeats originating from Xanthomonas or Ralstonia bacterial proteins AvrBs3, PthXo1, AvrHah1, PthA, Tal1c as non-limiting examples. These repeats differ essentially by two amino acids positions that specify an interaction with a base pair (Boch, J., H. Scholze, et al. (2009). “Breaking the code of DNA binding specificity of TAL-type III effectors.” Science 326(5959): 1509-12; Moscou, M. J. and A. J. Bogdanove (2009). “A simple cipher governs DNA recognition by TAL effectors.” Science 326(5959): 1501). Each base pair in the DNA target is contacted by a single repeat, with the specificity resulting from the two variant amino acids of the repeat (the so-called repeat variable dipeptide, RVD). TALE binding domains may further comprise an N-terminal translocation domain responsible for the requirement of a first thymine base (TO) of the targeted sequence and a C-terminal domain that containing a nuclear localization signals (NLS). A TALE nucleic acid binding domain generally corresponds to an engineered core TALE scaffold comprising a plurality of TALE repeat sequences, each repeat comprising a RVD specific to each nucleotides base of a TALE recognition site. In the present invention, each TALE repeat sequence of said core scaffold is made of 30 to 42 amino acids, more preferably 33 or 34 wherein two critical amino acids (the so-called repeat variable dipeptide. RVD) located at positions 12 and 13 mediates the recognition of one nucleotide of said TALE binding site sequence; equivalent two critical amino acids can be located at positions other than 12 and 13 specially in TALE repeat sequence taller than 33 or 34 amino acids long. Preferably, RVDs associated with recognition of the different nucleotides are HD for recognizing C, NG for recognizing T, NI for recognizing A, NN for recognizing G or A. In another embodiment, critical amino acids 12 and 13 can be mutated towards other amino acid residues in order to modulate their specificity towards nucleotides A, T, C and G and in particular to enhance this specificity. A TALE nucleic acid binding domain usually comprises between 8 and 30 TALE repeat sequences. More preferably, said core scaffold of the present invention comprises between 8 and 20 TALE repeat sequences; again more preferably 15 TALE repeat sequences. It can also comprise an additional single truncated TALE repeat sequence made of 20 amino acids located at the C-terminus of said set of TALE repeat sequences, i.e. an additional C-terminal half-TALE repeat sequence. Other modular base-per-base specific nucleic acid binding domains (MBBBD) are described in WO 2014/018601. Said MBBBD can be engineered, for instance, from newly identified proteins, namely EAV36_BURRH, E5AW43_BURRH, E5AW45_BURRH and E5AW46_BURRH proteins from the recently sequenced genome of the endosymbiont fungi Burkholderia Rhizoxinica. These nucleic acid binding polypeptides comprise modules of about 31 to 33 amino acids that are base specific. These modules display less than 40% sequence identity with Xanthomonas TALE common repeats and present more polypeptides sequence variability. The different domains from the above proteins (modules, N and C-terminals) from Burkholderia and Xanthomonas are useful to engineer new proteins or scaffolds having binding properties to specific nucleic acid sequences and may be combined to form chimeric TALE-MBBBD proteins.

### Methods and Compositions Related to Embodiments of the Invention

In certain aspects, the invention includes a method of making and/or expanding the antigen-specific redirected engineered immortalized CAR-T cells that comprises transfecting TCR/B2M deficient immortalized T cells with an expression vector containing a DNA construct encoding the CAR.

In another aspect, this invention is a method of stably transfecting and redirecting engineered immortalized T cells by electroporation, or other non-viral gene transfer (such as, but not limited to sonoporation) using naked DNA. Most investigators have used viral vectors to carry heterologous genes into T cells. By using naked DNA, the time required to produce redirected T cells can be reduced. “Naked DNA” means DNA encoding a chimeric T-cell receptor (cTCR) contained in an expression cassette or vector in proper orientation for expression. The electroporation method of this invention produces stable transfectants that express and carry on their surfaces the chimeric TCR (cTCR).

“Chimeric TCR” means a receptor that is expressed by T cells and that comprises intracellular signaling, transmembrane, and extracellular domains, where the extracellular domain is capable of specifically binding in an MHC unrestricted manner an antigen that is not normally bound by a T-cell receptor in that manner. Stimulation of the T cells by the antigen under proper conditions results in proliferation (expansion) of the cells. The exemplary BCMA and FN3 domain-specific chimeric receptors of this invention are examples of chimeric TCRs. However, the method is applicable to transfection with chimeric TCRs that are specific for other target antigens, such as chimeric TCRs that are specific for HER2/Neu, ERBB2, folate binding protein, renal cell carcinoma, and HIV-1 envelope glycoproteins gp20 and gp41. Other cell-surface target antigens include, but are not limited to, CD20, carcinoembryonic antigen, mesothelin, c-Met, CD56, HERV-K, GD2, GD3, aiphafetoprotein, CD23, CD30, CD123, IL-11Ralpha, kappa chain, lambda chain, CD70, CA-125, MUC-1, EGFR and variants, epithelial tumor antigen, and so forth.

In certain aspects, the T cells are immortalized human T cells. Conditions include the use of mRNA and DNA and electroporation. Following transfection, the cells may be immediately infused or may be stored. In certain aspects, following transfection, the cells may be propagated for days, weeks, or months ex vivo as a bulk population within about 1, 2, 3, 4, 5 days or more following gene transfer into cells. In a further aspect, following transfection, the transfectants are cloned and a clone demonstrating presence of a single integrated or episomally maintained expression cassette or plasmid, and expression of the chimeric receptor is expanded ex vivo. The clone selected for expansion demonstrates the capacity to specifically recognize and lyse BCMA-expressing target cells. The recombinant immortalized T cells may be expanded by stimulation with IL-2, or other cytokines that bind the common gamma-chain (e.g., IL-7, IL-15, IL-21, and others). In a further aspect, the genetically modified cells may be cryopreserved.

T-cell propagation (survival) after infusion may be assessed by: (i) q-PCR using primers specific for the CAR; and/or (ii) flow cytometry using an antibody specific for the CAR.

This invention also represents the targeting of a cancer, more particularly, multiple myeloma, with the cell-surface epitope being BCMA-specific using a redirected immortalized T cell that is devoid of TCR and B2M expression. Malignant B cells are an excellent target for redirected T cells, as B cells can serve as immunostimulatory antigen-presenting cells for T cells. In certain embodiments of the invention, the engineered immortalized T cells of the invention are delivered to an individual in need thereof, such as an individual that has cancer or an infection. The cells then enhance the individual's immune system to attack the respective cancer or pathogenic cells. In some cases, the individual is provided with one or more doses of the antigen-specific engineered immortalized T cells. In cases where the individual is provided with two or more doses of the antigen-specific engineered immortalized T cells, the duration between the administrations should be sufficient to allow time for propagation in the individual, and in specific embodiments the duration between doses is 1, 2, 3, 4, 5, 6, 7, or more days.

The source of the immortalized T cells that are modified to include both a chimeric antigen receptor and that lack functional TCRs and B2M may be of any kind, but in specific embodiments the cells are obtained from a bank of umbilical cord blood, peripheral blood, human embryonic stem cells, or induced pluripotent stem cells, for example. The different banks will not share the same HLAs, so multiple banks may be employed.

Suitable doses for a therapeutic effect would be at least 10⁵ or between about 10⁵ and about 10¹⁰ cells per dose, for example, preferably in a series of dosing cycles. An exemplary dosing regimen consists of four one-week dosing cycles of escalating doses, starting at least at about 105 cells on Day 0, for example increasing incrementally up to a target dose of about 10¹⁰ cells within several weeks of initiating an intra-patient dose escalation scheme. Suitable modes of administration include intravenous, subcutaneous, intracavitary (for example by reservoir-access device), intraperitoneal, and direct injection into a tumor mass.

A pharmaceutical composition of the present invention can be used alone or in combination with other well-established agents useful for treating cancer. Whether delivered alone or in combination with other agents, the pharmaceutical composition of the present invention can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

A composition of the present invention can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically acceptable diluent, carrier, or vehicle, where appropriate. The specifications for the novel unit dosage forms of the present invention depend on the particular pharmacodynamics associated with the pharmaceutical composition in the particular subject.

Desirably an effective amount or sufficient number of the engineered immortalized T cells is present in the composition and introduced into the subject such that long-term, specific, anti-tumor responses are established to reduce the size of a tumor or eliminate tumor growth or regrowth than would otherwise result in the absence of such treatment. Desirably, the amount of the engineered immortalized T cells reintroduced into the subject causes a 10%, 20%, 30%, 40%, 50%, 60%, 70%, 80%, 90%, 95%, 98%, or 100% decrease in tumor size when compared to otherwise same conditions wherein the engineered immortalized T cells are not present. Accordingly, the amount of the engineered immortalized T cells administered should take into account the route of administration and should be such that a sufficient number of the engineered immortalized T cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of the engineered immortalized T cells desirably should be sufficient to provide in the subject being treated at least from about 1×10⁶ to about 1×10⁹ engineered immortalized T cells, even more desirably, from about 1×10⁷ to about 5×10⁸ engineered immortalized T cells, although any suitable amount can be utilized either above, e.g., greater than 5×10′ cells, or below, e.g., less than 1×10⁷ cells. The dosing schedule can be based on well-established cell-based therapies (see, e.g., Topalian and Rosenberg, Ada Haematol, 78 Suppl 1:75-76, 1987; U.S. Pat. No. 4,690,915), or an alternate continuous infusion strategy can be employed.

These values provide general guidance of the range of the engineered immortalized T cells to be utilized by the practitioner upon optimizing the method of the present invention for practice of the invention. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation.

### Immune System and Immunotherapy

In some embodiments, a medical disorder is treated by transfer of a redirected immortalized T cell that elicits a specific immune response. In one embodiment of the present invention, a cancer or a medical disorder is treated by transfer of a redirected T immortalized T cell that elicits a specific immune response. Thus, a basic understanding of the immunologic responses is necessary.

The cells of the adaptive immune system are a type of leukocyte, called a lymphocyte. B cells and T cells are the major types of lymphocytes. B cells and T cells are derived from the same pluripotent hematopoietic stem cells, and are indistinguishable from one another until after they are activated. B cells play a large role in the humoral immune response, whereas T cells are intimately involved in cell-mediated immune responses. They can be distinguished from other lymphocyte types, such as B cells and NK cells by the presence of a special receptor on their cell surface called the T-cell receptor (TCR). In nearly all other vertebrates, B cells and T cells are produced by stem cells in the bone marrow. T cells travel to and develop in the thymus, from which they derive their name. In humans, approximately 1%-2% of the lymphocyte pool recirculates each hour to optimize the opportunities for antigen-specific lymphocytes to find their specific antigen within the secondary lymphoid tissues.

T lymphocytes arise from hematopoietic stem cells in the bone marrow, and migrate to the thymus gland to mature. T cells express a unique antigen binding receptor on their membrane (T-cell receptor), which can only recognize antigen in association with major histocompatibility complex (MHC) molecules on the surface of other cells. There are at least two populations of T cells, known as T helper cells and T cytotoxic cells. T helper cells and T cytotoxic cells are primarily distinguished by their display of the membrane bound glycoproteins CD4 and CD8, respectively. T helper cells secret various lymphokines that are crucial for the activation of B cells, T cytotoxic cells, macrophages, and other cells of the immune system. In contrast, T cytotoxic cells that recognize an antigen-MHC complex proliferate and differentiate into effector ceil called cytotoxic T lymphocytes (CTLs), CTLs eliminate cells of the body displaying antigen, such as virus infected cells and tumor cells, by producing substances that result in cell lysis. Natural killer cells (or NK cells) are a type of cytotoxic lymphocyte that constitutes a major component of the innate immune system. NK cells play a major role in the rejection of tumors and cells infected by viruses. The cells kill by releasing small cytoplasmic granules of proteins called perforin and granzyme that cause the target cell to die by apoptosis.

A B cell identifies pathogens when antibodies on its surface bind to a specific foreign antigen. This antigen/antibody complex is taken up by the B cell and processed by proteolysis into peptides. The B cell then displays these antigenic peptides on its surface MHC class II molecules. This combination of MHC and antigen attracts a matching helper T cell, which releases lymphokines and activates the B cell. As the activated B cell then begins to divide, its offspring (plasma cells) secrete millions of copies of the antibody that recognizes this antigen. These antibodies circulate in blood plasma and lymph, bind to pathogens expressing the antigen and mark (hem for destruction by complement activation or for uptake and destruction by phagocytes. Antibodies can also neutralize challenges directly, by binding to bacterial toxins or by interfering with the receptors used by viruses and bacteria to infect cells.

NK cells or natural killer cells are defined as large granular lymphocytes that do not express T-cell antigen receptors (TCR) or Pan T marker CDS or surface immunoglobulins (Ig) B cell receptor but that usually express the surface markers CD16 (FcγRIII) and CD56 in humans, and NK1.1/NK1.2 in certain strains of mice.

Antigen-presenting cells, which include macrophages, B lymphocytes, and dendritic cells, are distinguished by their expression of a particular MHC molecule. APCs internalize antigen and re-express a part of that antigen, together with the MHC molecule on their outer cell membrane. The major histocompatibility complex (MHC) is a large genetic complex with multiple loci. The MHC foci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans, the MHC is referred to as the HLA complex and in mice the H-2 complex.

The T-cell receptor, or TCR, is a molecule found on the surface of T lymphocytes (or T cells) that is generally responsible for recognizing antigens bound to major histocompatibility complex (MHC) molecules. It is a heterodimer consisting of an alpha and beta chain in 95% of T cells, while 5% of T cells have TCRs consisting of gamma and delta chains. Engagement of the TCR with antigen and MHC results in activation of its T lymphocyte through a series of biochemical events mediated by associated enzymes, co-receptors, and specialized accessory molecules. In immunology, the CDS antigen (CD stands for cluster of differentiation) is a protein complex composed of four distinct chains (CDSv, CD35, and two times CDSe) in mammals, that associate with molecules known as the T-cell receptor (TCR) and the l-chain to generate an activation signal in T lymphocytes. The TCR, c-chain, and CDS molecules together comprise the TCR complex. The CD3y, CD38, and CD3s chains are highly related cell surface proteins of the immunoglobulin superfamily containing a single extracellular immunoglobulin domain. The transmembrane region of the CDS chains is negatively charged, a characteristic that allows these chains to associate with the positively charged TCR chains (TCRa and TCRfi). The intracellular tails of the CDS molecules contain a single conserved motif known as an immunoreceptor tyrosine-based activation motif or IT AM for short, which is essential for the signaling capacity of the ‘T’CR.

CD28 is one of the molecules expressed on T cells that provide co-stimulatory signals, which are required for T cell activation. CD28 is the receptor for B7.1 (CD80) and B7.2 (CD86). When activated by Toil-like receptor ligands, the B7.1 expression is upregulated in antigen presenting ceils (APCs). The B7.2 expression on antigen presenting cells is constitutive. CD28 is the only B7 receptor co stitutively expressed on naive T cells. Stimulation through CD28 in addition to the TCR can provide a potent co-stimulatory signal to T cells for the production of various interleukins (IL-2 and IL-6 in particular).

The strategy of isolating and expanding antigen-specific T cells as a therapeutic intervention for human disease has been validated in clinical trials (Riddell et al, Science, 257:238, 1992, 1992; Walter et al, N. Engl. J. Med., 333: 1038, 1995; Heslop et al., Nat. Med., 2:551, 1996)

Malignant B cells appear to be excellent targets for redirected T cells, as B cells can serve as immunostimulatory antigen-presenting cells for T cells (Glimcher et al, J. Exp. Med, 155:445, 1982). Lymphoma, by virtue of its lymph node tropism, is anatomically ideally situated for T cell-mediated recognition and elimination. The localization of infused T cells to lymph node in large numbers has been documented in HIV patients receiving infusions ofHIV-specific CD8⁺ CTL clones. In these patients, evaluation of lymph node biopsy material revealed that infused clones constituted approximately 2%-8% of CD8+ cells of lymph nodes. Lymph node homing might be further improved by co-transfecting T cells with a cDNA construct encoding the L-selection molecule under a constitutive promoter since this adhesion molecule directs circulating T cells back to lymph nodes and is down-regulated by in vitro expansion (Chao et al, J. Immunol, 159: 1686, 1997). The present invention may provide a method of treating a human disease condition associated with a cell expressing endogenous BCMA comprising infusing a patient with a therapeutically effective dose of the recombinant human BCMA-specific CAR expressing cell as described above. The human disease condition associated with a cell expressing endogenous BCMA may be selected from the group consisting of multiple myeloma, lymphoma, leukemia, non-Hodgkin's lymphoma, acute lymphoblastic leukemia, chronic lymphoblastic leukemia, chronic lymphocytic leukemia, and B cell-associated autoimmune diseases.

Multiple myeloma (MM) is a cancer that is characterized by an accumulation of clonal plasma cells. MM is the second most common hematologic malignancy, and it accounts for as many as 2% of deaths from all cancers. MM is a heterogeneous disease and is characterized by a wide range of aggression and treatment resistance. Some patients live a decade or longer after diagnosis, while others suffer rapid treatment-resistant progression and die within 2 years. Despite progress in the development of new therapeutics, there is currently no cure for MM. Though current therapies often lead to remission of MM, the disease eventually relapses in nearly all patients and is ultimately fatal (Naymagon and Abdul-Hay, J Hematol Oncol. 2016 Jun. 30; 9(1):52). In addition, traditional methods of treatment, including chemotherapy and radiation therapy, have limited utility due to toxic side effects

Leukemia is a cancer of the blood or bone marrow and is characterized by an abnormal proliferation (production by multiplication) of blood cells, usually white blood cells (leukocytes). t is part of the broad group of diseases called hematological neoplasms. Leukemia is a broad term covering a spectrum of diseases. Leukemia is clinically and pathologically split into its acute and chronic forms.

Acute leukemia is characterized by the rapid proliferation of immature blood cells. This crowding makes the bone marrow unable to produce healthy blood cells. Acute forms of leukemia can occur in children and young adults. In fact, it is a more common cause of death for children in the U.S. than any other type of malignant disease. Immediate treatment is required in acute leukemia due to the rapid progression and accumulation of the malignant cells, which then spill over into the bloodstream and spread to other organs of the body. Central nervous system (CNS) involvement is uncommon, although the disease can occasionally cause cranial nerve palsies. Chronic leukemia is distinguished by the excessive build-up of relatively mature, but still abnormal, blood cells. Typically taking months to years to progress, the cells are produced at a much higher rate than normal cells, resulting in many abnormal white blood cells in the blood. Chronic leukemia mostly occurs in older people, but can theoretically occur in any age group. Whereas acute leukemia must be treated immediately, chronic forms are sometimes monitored for some time before treatment to ensure maximum effectiveness of therapy.

Furthermore, the diseases are classified into lymphocytic or lymphoblastic, which indicate that the cancerous change took place in a type of marrow cell that normally goes on to form lymphocytes, and myelogenous or myeloid, which indicate that the cancerous change took place in a type of marrow ceil that normally goes on to form red cells, some types of white cells, and platelets (see lymphoid cells vs. myeloid cells).

Acute lymphocytic leukemia (also known as acute lymphoblastic leukemia, or ALL) is the most common type of leukemia in young children. This disease also affects adults, especially those aged 65 and older. Chronic lymphocytic leukemia (CLL) most often affects adults over the age of 55. It sometimes occurs in younger adults, but it almost never affects children. Acute myelogenous leukemia (also known as acute myeloid leukemia, or AML) occurs more commonly in adults than in children. This type of leukemia was previously called “acute nonlymphocytic leukemia.” Chronic myelogenous leukemia (CML) occurs mainly in adults, A very small number of children also develop this disease.

Lymphoma is a type of cancer that originates in lymphocytes (a type of white blood cell in the vertebrate immune system). There are many types of lymphoma. According to the U.S. National Institutes of Health, lymphomas account for about five percent of all cases of cancer in the United States, and Hodgkin's lymphoma in particular accounts for less than one percent of all cases of cancer in the United States. Because the lymphatic system is pari of the body's immune system, patients with a weakened immune system, such as from HIV infection or from certain drags or medication, also have a higher incidence of lymphoma.

In the 19th and 20th centuries the affliction was called Hodgkin's Disease, as it was discovered by Thomas Hodgkin in 1832. Colloquially, lymphoma is broadly categorized as Hodgkin's lymphoma and non-Hodgkin lymphoma (all other types of lymphoma). Scientific classification of the types of lymphoma is more detailed. Although older classifications referred to histiocytic lymphomas, these are recognized in newer classifications as of B, T, or NK cell lineage.

Autoimmune disease, or autoimmunity, is the failure of an organism to recognize its own constituent parts (down to the sub-molecular levels) as “self,” which results in an immune response against its own cells and tissues. Any disease that results from such an aberrant immune response is termed an autoimmune disease. Prominent examples include Coeliac disease, diabetes mellitus type 1 (IDDM), systemic lupus erythematosus (SLE), Sjogren's syndrome, multiple sclerosis (MS), Hashimoto's thyroiditis, Graves' disease, idiopathic thrombocytopenic purpura, and rheumatoid arthritis (EA).

Inflammatory diseases, including autoimmune diseases are also a class of diseases associated with B-cell disorders. Examples of autoimmune diseases include, but are not limited to, acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, peraiciousanemia, rapidly progressive glomerulonephritis, psoriasis, and fibrosing alveolitis. The most common treatments are corticosteroids and cytotoxic drugs, which can be very toxic. These drugs also suppress the entire immune system, can result in serious infection, and have adverse effects on the bone marrow, liver, and kidneys. Other therapeutics that has been used to treat Class III autoimmune diseases to date have been directed against T cells and macrophages. There is a need for more effective methods of treating autoimmune diseases, particularly Class III autoimmune diseases.

### Embodiments of Kits of the Invention

Any of the compositions described herein may be comprised in a kit. in some embodiments, engineered immortalized CAR-T cells are provided in the kit, which also may include reagents suitable for expanding the cells, such as media.

In a non-limiting example, a chimeric receptor expression construct, one or more reagents to generate a chimeric receptor expression construct, cells for transfection of the expression construct, and/or one or more instruments to obtain immortalized T cells for transfection of the expression construct (such an instrument may be a syringe, pipette, forceps, and/or any such medically approved apparatus).

In some embodiments, an expression construct for eliminating endogenous TCR expression and B2M, one or more reagents to generate the construct, and/or CAR+ T cells are provided in the kit.

In some embodiments, there includes expression constructs that encode Cas9 endonucleases.

In some aspects, the kit comprises reagents or apparatuses for electroporation of cells.

In some embodiments, the kit comprises artificial antigen presenting cells.

The kits may comprise one or more suitably aliquoted compositions of the present invention or reagents to generate compositions of the invention. The components of the kits may be packaged either in aqueous media or in lyophilized form. The container means of the kits may include at least one vial, test tube, flask, bottle, syringe, or other container means, into which a component may he placed, and preferably, suitably aliquoted. Where there is more than one component in the kit, the kit also will generally contain a second, third, or other additional container into which the additional components may be separately placed. However, various combinations of components may be comprised in a vial. The kits of the present invention also will typically include a means for containing the chimeric receptor construct and any other reagent containers in close confinement for commercial sale. Such containers may include injection or blow molded plastic containers into which the desired vials are retained, for example.

### Embodiments

The invention also provides the following non-limiting embodiments.
1. An engineered immortalized T cell line expressing a chimeric antigen receptor (CAR), comprising:
   (a) an extracellular domain comprising an antigen binding region;
   (b) a transmembrane domain; and
   (c) an intracellular signaling domain,
wherein the immortalized T cell line does not express at least one endogenous T cell receptor and does not express beta 2-microglobulin (B2M).
2. The immortalized T cell line of embodiment 1, wherein the antigen binding region binds a tumor associated antigen.
3. The immortalized T cell line of embodiment 2, wherein the tumor associated antigen is BCMA.
4. The immortalized T cell line of embodiment 1, wherein the antigen binding region binds a fibronectin type III (FN3) domain.
5. The immortalized T cell line of embodiment 1, wherein the at least one endogenous T cell receptor is knocked out.
6. The immortalized T cell line of embodiment 1, wherein the at least one endogenous T cell receptor is TCR-alpha.
7. The immortalized T cell line of embodiment 1, wherein the at least one endogenous T cell receptor is KIR3DL2.
8. The immortalized T cell line of embodiment 1, wherein B2M is knocked out.
9. An engineered TALL-104 cell line expressing a CAR, comprising:
   (a) an extracellular domain comprising an antigen binding region;
   (b) a transmembrane domain; and
   (c) an intracellular signaling domain,
wherein the TALL-104 cell line does not express at least one endogenous T cell receptor and does not express beta 2-microglobulin (B2M).
10. The cell line of embodiment 9, wherein the antigen binding region binds a tumor associated antigen.
11. The cell line of embodiment 10, wherein the tumor associated antigen is BCMA.
12. The cell line of embodiment 9, wherein the antigen binding region binds a fibronectin type III (FN3) domain.
13. The cell line of embodiment 9, wherein the at least one endogenous T cell receptor is knocked out.
14. The cell line of embodiment 9, wherein the at least one endogenous T cell receptor is TCR-alpha.
15. The cell line of embodiment 9, wherein the at least one endogenous T cell receptor is KIR3DL2.
16. The cell line of embodiment 9, wherein B2M is knocked out.
17. An engineered TALL-104 cell line expressing a CAR, comprising:
   (a) a signal peptide having an amino acid sequence of SEQ ID NO: 3;
   (b) an extracellular domain comprising an FN3 domain having an amino acid sequence of any one of SEQ ID NOs: 8-44;
   (c) a hinge region having an amino acid sequence of SEQ ID NO: 4;
   (d) a transmembrane domain having an amino acid sequence of SEQ ID NO: 5; and
   (e) an intracellular signaling domain comprising a co-stimulatory domain having an amino acid sequence of SEQ ID NO: 6, and a primary signaling domain having an amino acid sequence of SEQ ID NO: 7;
wherein the cell line does not express TRCA, KIR3DL2 and B2M.
18. An engineered TALL-104 cell line expressing a CAR, comprising:
   (a) an extracellular domain comprising an scFv having an amino acid sequence of any one of SEQ ID NOs: 54 and 55;
   (b) a hinge region having an amino acid sequence of SEQ ID NO: 4;
   (c) a transmembrane domain having an amino acid sequence of SEQ ID NO: 5; and
   (d) an intracellular signaling domain comprising a co-stimulatory domain having an amino acid sequence of SEQ ID NO: 6, and a primary signaling domain having an amino acid sequence of SEQ ID NO: 7.
      wherein the TALL-104 cell line does not express TRCA, KIR3DL2 and B2M.
19. An in vitro method of generating an engineered immortalized T cell line expressing a CAR, comprising the steps of:
   a. providing an immortalized T cell line;
   b. inhibiting the expression of at least one endogenous T cell receptor and B2M; and
   c. introducing a polynucleotide that encodes a CAR into the immortalized T cell.
20. The method of embodiment 19, wherein step b occurs before step c.
21. The method of embodiment 19, wherein step c occurs before step b.
22. The method of embodiment 19, wherein step b is performed by using an endonuclease.
23. The method of embodiment 22, where in the endonuclease is a TAL-nuclease, meganuclease, zing-finger nuclease (ZFN), or Cas9.
24. The method of embodiment 19, wherein step c is further defined as introducing a polynucleotide that encodes a CAR into the immortalized T cell by electroporation or a viral-based gene transfer system.
25. The method of embodiment 4, wherein the viral-based gene transfer system comprises a retroviral vector, adenoviral vector, adeno-associated viral vector, or lentiviral vector.
26. A pharmaceutical composition, comprising the engineered immune cell of any of embodiments 1-18 and a pharmaceutically acceptable carrier.
27. A method of treating a cancer in a subject in need thereof, comprising administering to the subject a therapeutically effective amount of the pharmaceutical composition of embodiment 26.
28. The method of embodiment 27, wherein the cancer is multiple myeloma.
29. A method of producing a pharmaceutical composition, comprising combining the engineered immortalized T-cell lines of any of embodiments 1-18 with a pharmaceutically acceptable carrier to obtain the pharmaceutical composition.

### EXAMPLES

The following examples of the invention are to further illustrate the nature of the invention. It should be understood that the following examples do not limit the invention and that the scope of the invention is to be determined by the appended claims.

### Example 1: Preparation of TALL-104 Cells for Electroporation

Exponentially growing TALL-104 cells were seeded at a density of 0.7×10⁶ cells/mL in Complete TALL-104 cell media [Myelocult H5100 Media (StemCell Technologies 05150); 1% Sodium Pyruvate (Invitrogen 11360-070); 1% Non-Essential Amino Acids (Invitrogen 11140-050); 4 uM Hydrocortisone (StemCell Technologies 07904); 100 IU/ml recombinant human IL-2 (R&D Systems 202-IL, 2.1E4 IU/ug)] and incubated at 37° C. The following day, the desired number of cells (1×10⁶/electroporation) were collected by centrifugation at 100×g for 10 min. Cells were washed twice with 10 mL of cold Opti-MEM (ThermoFisher Scientific, 31985062), centrifuged at 100×g for 10 min and re-suspended in 0.1 mL×(total number of electroporation experiments+1) of OPTI-MEM previously equilibrated to room temperature.

### Example 2: Preparation of Ribonucleoprotein Complexes

### Guide RNA

A gRNA was designed to target the first exon of the constant chain of the TCRα gene (TRAC). The sequence targeted, located upstream of the transmembrane domain of TCRα, is required for the TCRα and β assembly and addressing to the cell-surface. Upon Cas9 endonuclease-mediated DNA cleavage, either non-homologous end joining (NHEJ) or integration of the CAR by homology directed repair (HDR) would result in ablation of the TRAC gene. For disruption of the B2M locus, a gRNA was designed targeting the first exon. For the KIR3DL2, a gene responsible for producing trans-membrane glycoproteins on natural killer cells and subsets of T cells, a gRNA was designed targeting the third exon.

TABLE 1
Sequence of Guide RNAs for gene editing.

| Guide RNA | gRNA sequence | | | |
|---|---|---|---|---|
| target (gRNA) | (Protospacer) | PAM | Strand | Exon |
| TCRa #1 | GCUGGUACACGGCAGGGU | GGG | — | 1 |
| | CA (SEQ ID NO: 56) | | | |
| TCRa #2 | GAGAAUCAAAAUCGGUGA | AGG | — | 1 |
| | AU (SEQ ID NO: 57) | | | |
| B2M-L | CCGGUGCCUCGCUCUGUA | GCC | — | 1 |
| | GA (SEQ ID NO: 58) | | | |
| B2M-R | ACUCUCUCUUUCUG CCU | AGG | — | 1 |
| | GG (SEQ ID NO: 59) | | | |
| KIR3DL2 #2 | AGAGCCACGUGUCC CCU | AGG | — | 3 |
| | CG (SEQ ID NO: 60) | | | |
| KIR3DL2 #3 | UCUCCUGGAAUAUUCUGC | TGG | — | 3 |
| | CG (SEQ ID NO: 61) | | | |

Formation of the gRNA:tracrRNA Duplex

Target-specific Alt-R CRISPR-Cas9 guide RNAs (gRNAs) were custom-synthesized by Integrated DNA Technologies. The universal 67mer Alt-R™ CRISPR-Cas9 tracrRNA (1072534) that hybridizes to the gRNA was obtained from Integrated DNA Technologies. Alt-R™ CRISPR-Cas9 gRNA and Alt-R™ CRISPR-Cas9 tracrRNA were re-suspended in IDTE Buffer (Integrated DNA Technologies, 11-01-03-01) to a final concentration of 200 μM. The two RNA oligos were mixed at equimolar concentrations in a sterile microcentrifuge tube to a final duplex concentration of 100 μM. The gRNA:tracrRNA mixture was heated for 5 min at 95° C. after which it was allowed to cool to room temperature on the benchtop to facilitate duplex formation.

### Formation of the Ribonucleoprotein (RNP) Complex

In a sterile PCR tube, add 2.1 uL of PBS, 1.2 uL (120 pmol) of the gRNA:tracrRNA duplex and 1.7 ul (104 pmol) of Alt-R S. pyogenes Cas9 enzyme (Integrated DNA Technologies, 1078728) Mix and incubate for 20 mins at room temperature to allow for RNP formation.

### Example 3: Electroporation of TALL-104 Cells with RNP Complexes

Five μL of the RNP complex and 0.1 μL (1×10̂6 cells) of prepared TALL-104 cells were added into a 2 mm gap size BTX electroporation cuvette (BTX, 45-0135). The cells were electroporated with a single pulse at 200 V for 10 milliseconds using the ECM 830 Square Wave Electroporation System (BTX) per the manufacturer's protocol. The electroporated cells were immediately transferred into one 12-well plate containing TALL-104 cell media previously equilibrated at 37° C. The media was replaced 24 hours post-electroporation.

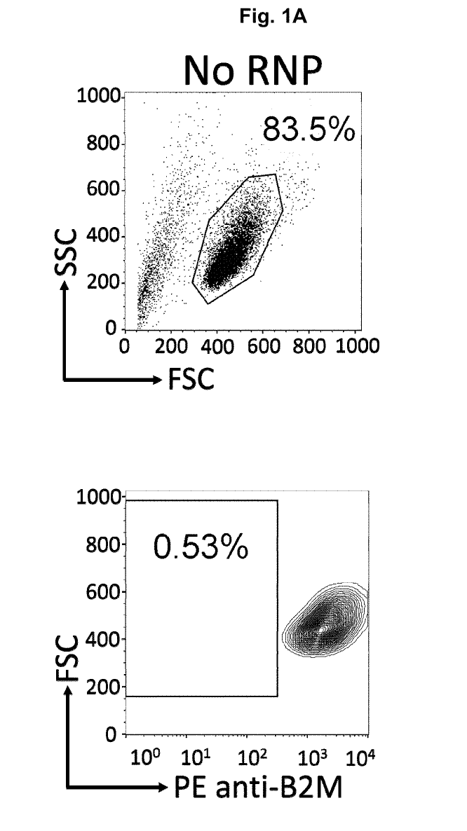

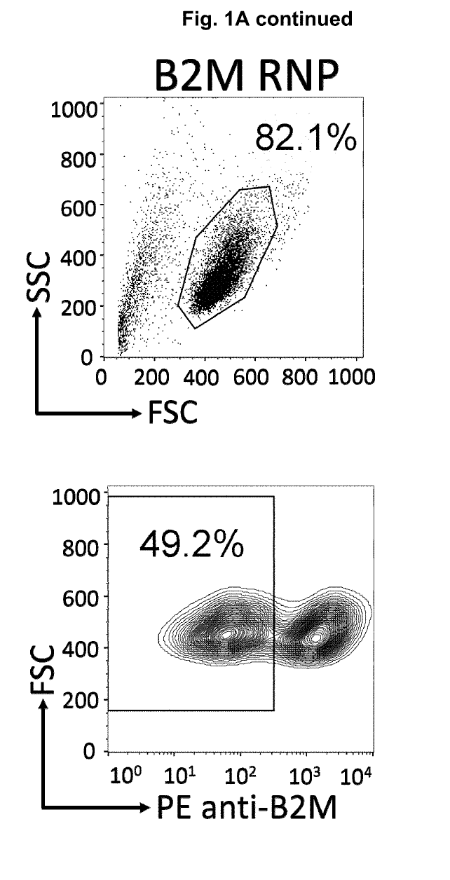

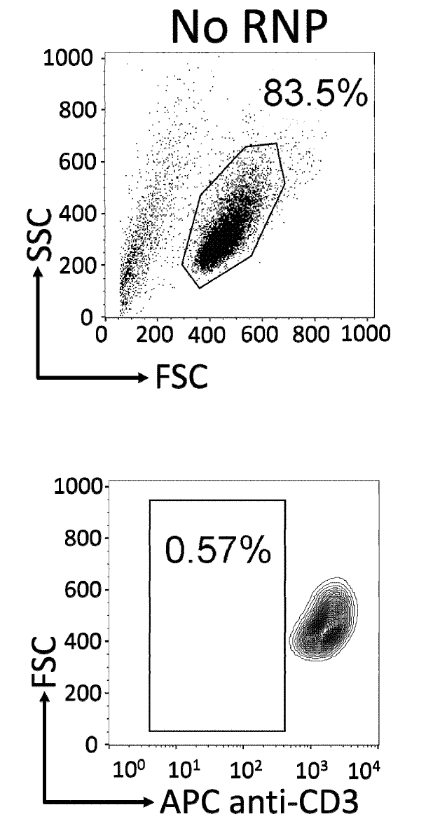

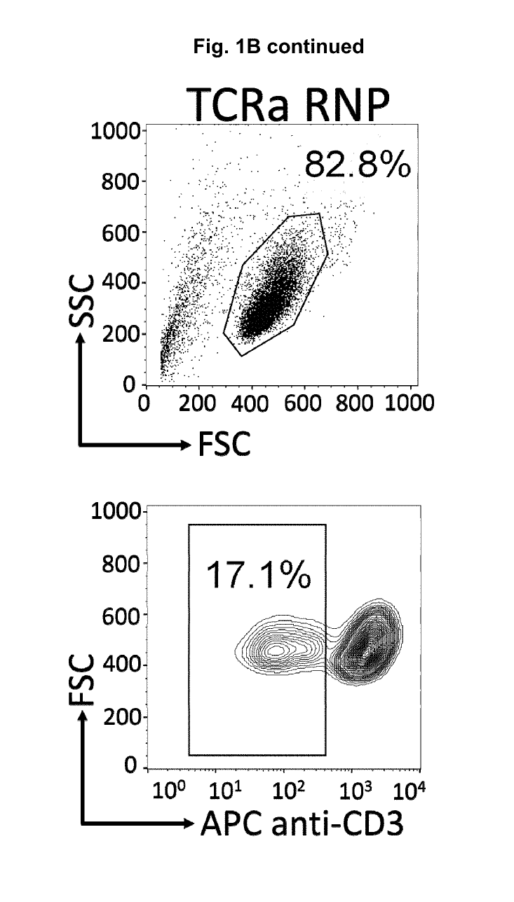

The efficiency of CRISPR-Cas9-mediated gene editing was analyzed by flow cytometry on either the FACS Calibur or LSRFortessa (BD Biosciences). 100,000 cells were harvested 5 days post-electroporation of the RNP complex by centrifugation at 100×g for 10 mins. Cells were washed 2× with 200 uL of stain buffer (BD Biosciences, 554657), and re-suspended in 100 uL of stain buffer. The relevant antibodies (PE-labeled mouse anti-human Beta 2 Microglobulin (B2M) antibody (BD Pharmingen. 551337) or isotype control antibody (Biolegend, 400214), APC-labeled mouse anti-human CD3 antibody (Biolegend. 300439) or isotype control (Biolegend, 400120) according to manufacturer's instructions and incubated at 4° C. in the dark for 45 mins. The cells were centrifuged at 100×g, washed 2× with stain buffer and re-suspended in 200 uL of stain buffer. Data was collected by flow cytometry and analyzed using FloJo software. FIG. 1 shows the levels of B2M and TCR knock-out sub-population after electroporating with the relevant RNP complexes.

### Example 4: Isolation of Gene-Edited TALL-104 Sub-Population Post-CRISPR CAS9-Mediated Editing

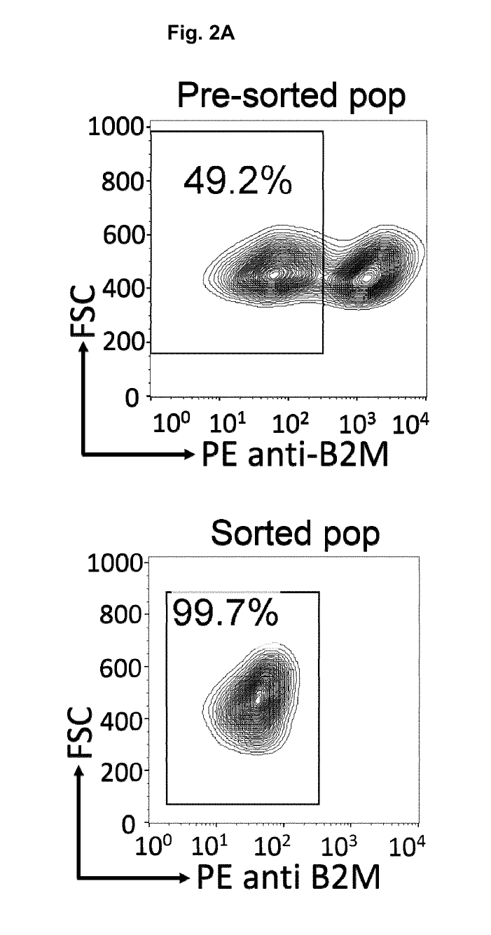

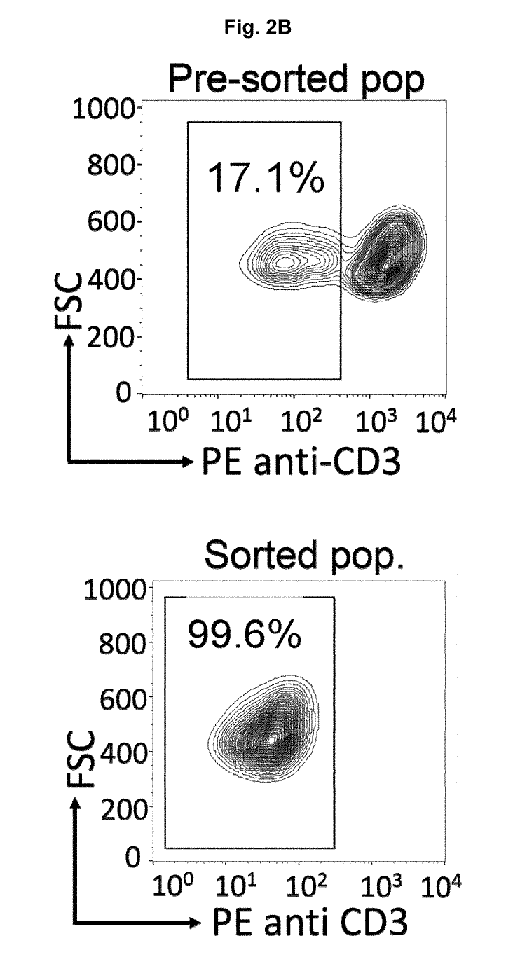

Gene-edited TALL-104 cells were isolated from non-edited wild type cells by magnetic cell separation (MACS) technology, using magnetic beads coated with anti-Phycoerythrin monoclonal antibody (mAb) Briefly, TALL-104 cells were counted, centrifuged at 100×g for 10 min at 4° C. and washed twice in 5 mL of cold. de-gassed Buffer X (PBS containing 0.5% BSA and 2 mM EDTA). Cells were re-suspended in 1 mL of Buffer X and incubated at 4° C. for 45-60 min with a PE-conjugated antibody targeting the protein of interest according to manufacturer's instructions (PE anti-CD3 or PE anti-B2M). The cells were centrifuged at 100×g for 10 min at 4° C. and re-suspended in 0.5 mL of cold buffer X containing anti-PE microbeads (Miltenyi Biotec, Cat#130-105-639). The mixture was incubated at 4° C. in the dark for 30 mins, centrifuged and re-suspended in 500 μL Buffer X. The cells were loaded onto a LS column (Miltenyi Biotec 130-042-401) placed on a QuadroMACS separator (Miltenyi Biotec, 130-090-976) previously equilibrated with 3 mL of Buffer X. The column was washed two times with 1 mL of Buffer X. Gene-edited knock out TALL-104 cells were isolated and collected in the flow through, and cultured in TALL-104 Complete cell media (0.7×10⁶ cells/ml) at 37*C and 5% CO2. FIG. 2 shows the isolation of the gene-edited knockout cells using MACS magnetic bead labeled technology.

### Example 5: Generation and Analysis of Engineered TALL-104 Cells Expressing Cars Targeting BCMA or Fibronectin Type III Domains

The amino acid sequence of the two different CAR sequences (were back-translated and engineered with signal peptide, hinge sequence, TM domain, and signaling domains. The completed construct was cloned into a T7 in vitro transcription vector to generate mRNA using the commercially available mMESSAGE mMACHINE® T7 ULTRA Transcription Kit.

TABLE 2
Amino Acid Sequence of the D08 CAR construct which
features an extracellular FN3 domain that can
target BCMA.

| Domain | Sequence |
|---|---|
| human CD8 | SEQ ID NO: 3 |
| signal | MALPVTALLLPLALLLHAARP |
| peptide | |
| human CD8 | SEQ ID NO: 4 |
| hinge | TTTPAPRPPTPAPTIASQPLSLRPEACRPAAGGAVH |
| | TRGLDFACDIY |
| extracellular | SEQ ID NO: 14 (D08) |
| BCMA- | MLPAPKNLVVSHVTEDSARLSWTAPDAAFDSFIIVY |
| specific FN3 | RENIETGEAIVLTVPGSERSYDLTDLKPGTEYYVQI |
| domain | AGVKGGNISFPLSAIFTT |
| human CD8 | SEQ ID NO: 5 |
| TM domain | IWAPLAGTCGVLLLSLVITLYCK |
| human 4- | SEQ ID NO: 6 |
| 1BB | RGRKKLLYIFKQPFMRPVQTTQEEDGCSCRFPEEEE |
| intracellular | GGCEL |
| domain | |
| human CD3 | SEQ ID NO: 7 |
| zeta | RVKFSRSADAPAYKQGQNQLYNELNLGRREEYDVLD |
| intracellular | KRRGRDPEMGGKPRRKNPQEGLYNELQKDKMAEAYS |
| domain | EIGMKGERRRGKGHDGLYQGLSTATKDTYDALHMQA |
| | LPPR |

TABLE 3
Amino Acid Sequence of the A57B91 CAR construct
which targets FN3 domains.

| Domain | Sequence |
|---|---|
| Extra- | SEQ ID NO: 55 (L-H orientation) |
| cellular | DVVMTQTPASVSGPVGGTVTIKCQASERIYSNLAWYQQ |
| AS7B91 | KPGQPPKLLIYKASTLASGVSSRFKGSGSGTEFTLTIR |
| | DLECADAATYSCQYTSYGSGYVGTFGGGTEVVVEGGGG |
| | GSGGGGSGGGGSGGGGSLEESGGRLVTPGTPLTLTCTV |
| | SGIDLSTSVMGWVRQAPGKGLESIGFIYTNVNTYYASW |
| | AKGRFTISRTSTTVDLKITSPTTGDTATYFCARAVYAG |
| | AMDLWGQGTLVTVSS |
| human CD8 | SEQ ID NO: 4 |
| hinge | TTTPAPRPPTPAPTIASQPLSLRPEACRPAAGGAVHTR |
| | GLDFACDIY |
| human CD8 | SEQ ID NO: 5 |
| TM domain | IWAPLAGTCGVLLLSLVITLYCK |
| human 4- | SEQ ID NO: 6 |
| 1BB | RGRKKLLYIFKQPFMRPVQTTQEEDGCSCRFPEEEEGG |
| intra- | CEL |
| cellular | |
| domain | |
| human CD3 | SEQ ID NO: 7 |
| zeta | RVKFSRSADAPAYKQGQNQLYNELNLGRREEYDVLDKR |
| intra- | RGRDPEMGGKPRRKNPQEGLYNELQKDKMAEAYSEIGM |
| cellular | KGERRRGKGHDGLYQGLSTATKDTYDALHMQALPPR |
| domain | |

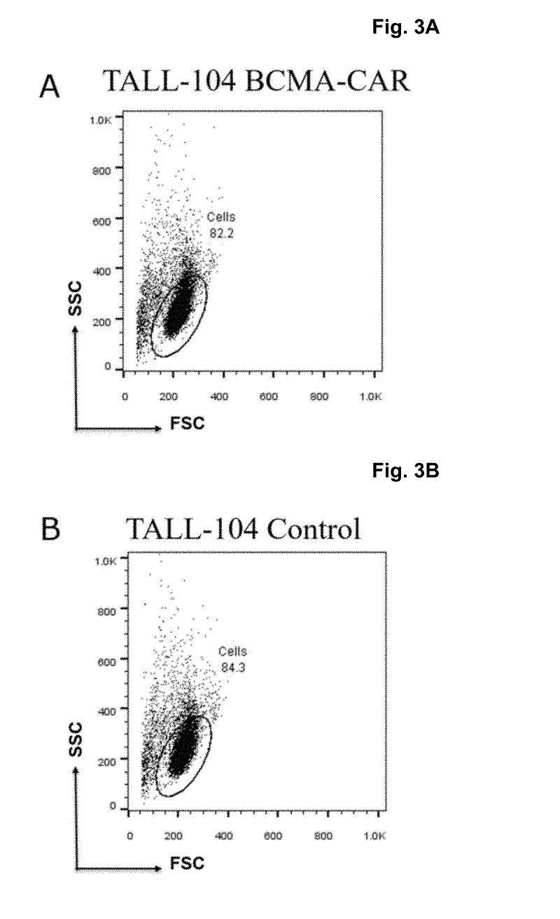

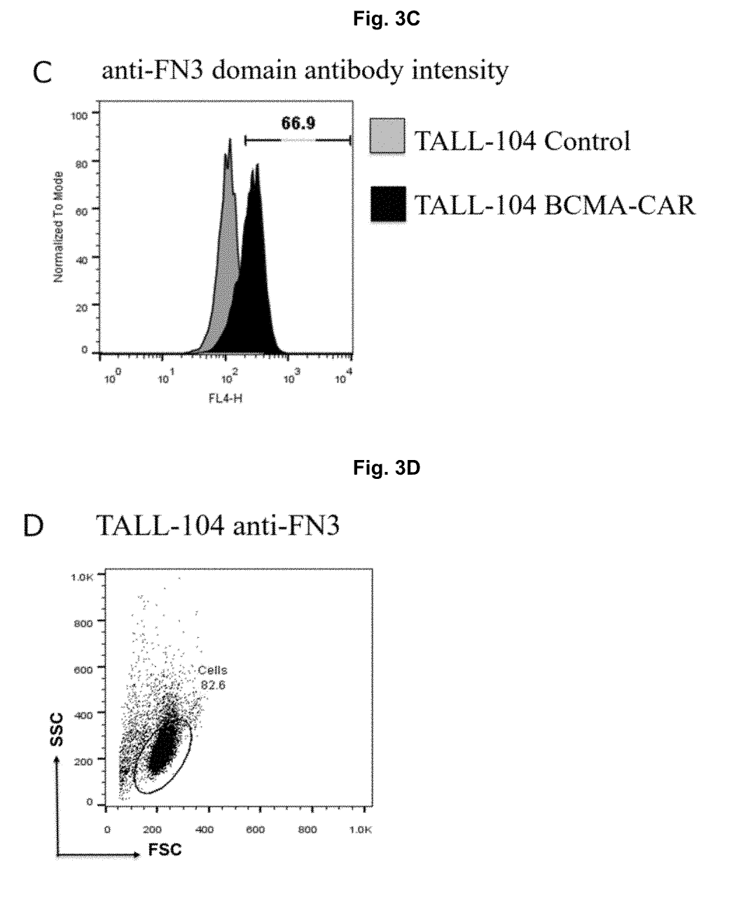

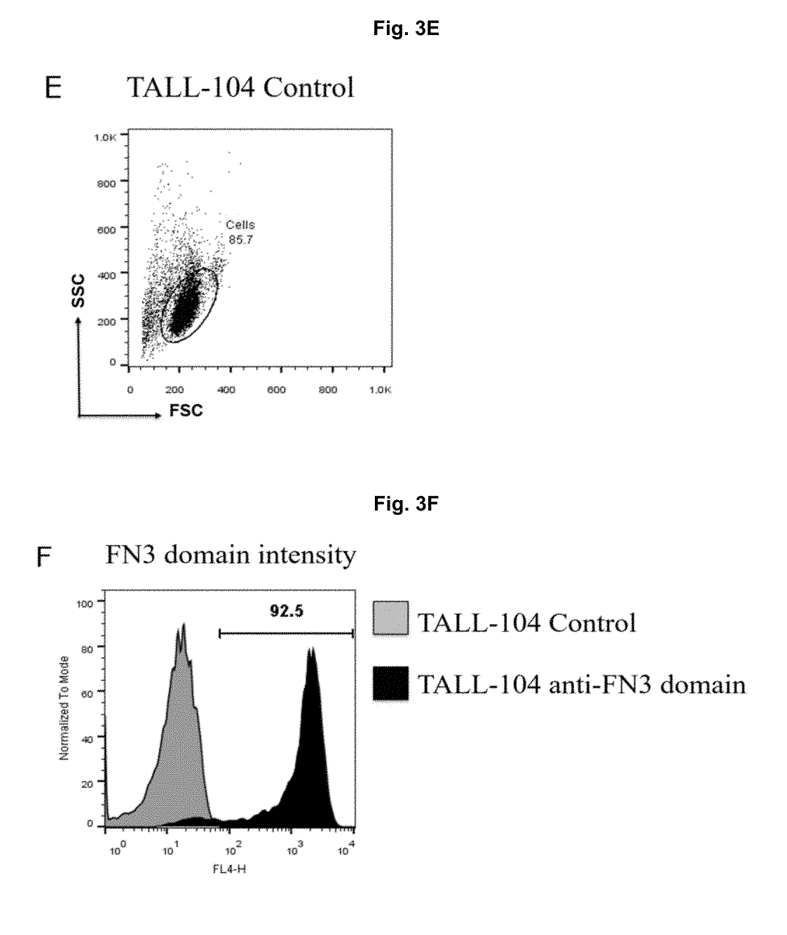

mRNA was electroporated into TALL-104 cells using the ECM 830 Square Wave Electroporation System (BTX). 3.5×10⁶ TALL-104 cells, which had been growing for three weeks in Complete TALL-104 media, received a single electric pulse (400V, 750 us) per the manufacturer's protocol, either with or without 10 jpg of CAR mRNA. Surface expression of the D08 CAR was assessed 24 hours later using AS7B91 anti-FN3 domain antibody. Similarly, the surface expression of the AS7B91 CAR was assessed 24 hours later using a conjugated FN3 domain. The results shown in FIG. 3 demonstrate that TALL-104 express the BCMA and anti-FN3 domain CARs.

### Example 6: Cytotoxicity Assay Using TALL-104 Engineered Cells as Effector Cells

BCMA-targeting (D08) and FN3 domain-targeting (AS7B91) CAR-TALL-104 cell killing was evaluated using as targets cells BCMA-expressing and CellTracker™ green-stained RPMI-8226 cells (ATCC: CCL-155), Daudi cells (ATCC: CCL-213), and K562 cells (ATCC: CCL-243)—all of which express BCMA at varying levels. AS7B91-CAR-TALL-104 cells, D08-CAR-TALL-104 cells, and mock TALL-104 cells (no mRNA electroporated) were coincubated for 20 hours with the BCMA target cells at an E:T ratio of ˜0.2 per well. For AS7B91-CAR-TALL-104 cells, a BCMA-specific or non-targeted control (NT) FN3 domain was coincubated with the cells. At the end of the experiment, cells were stained for 15 minutes with HOECHST 33342 (nucleus) stain plus Propidium Iodide (dead cell stain).

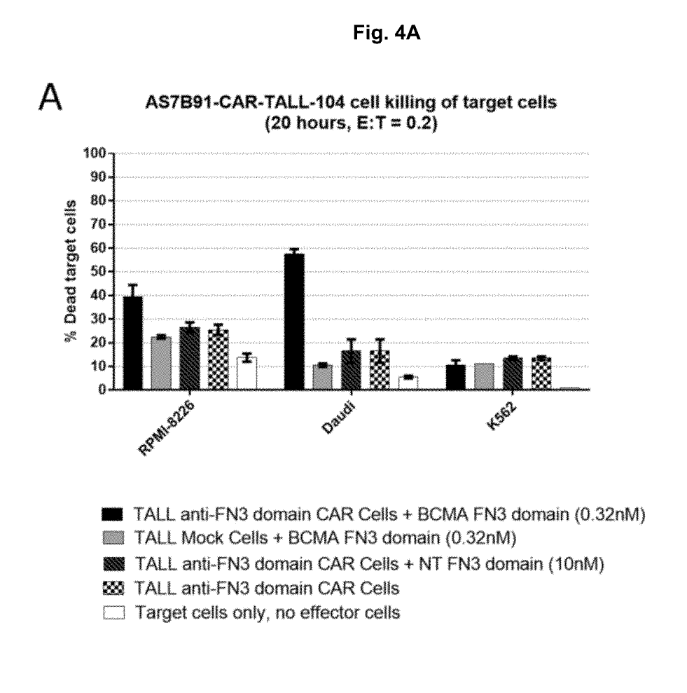

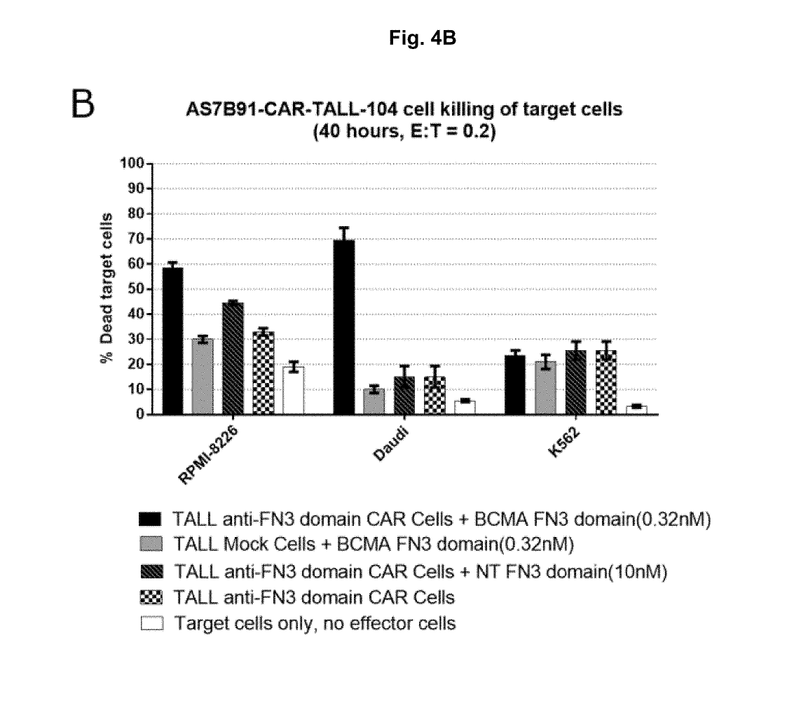

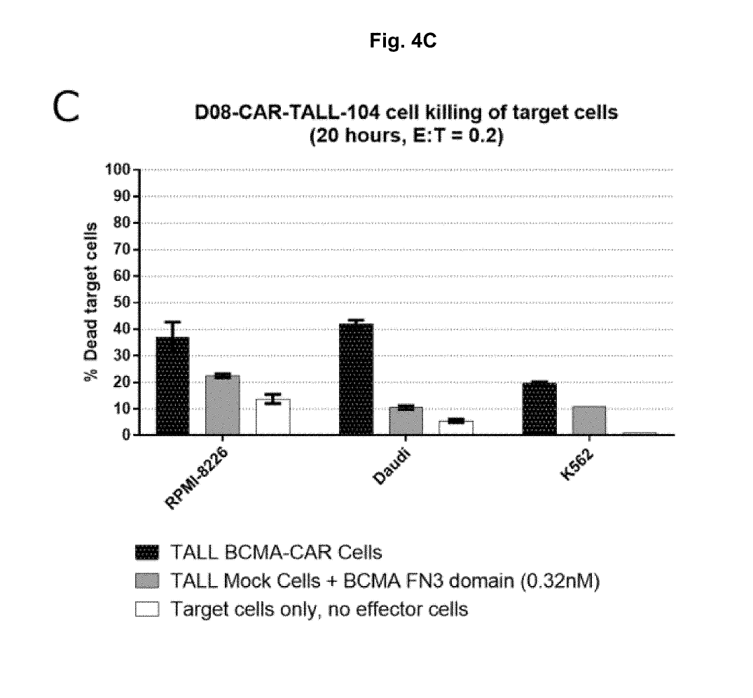

The cells were imaged on a PerkinElmer Opera confocal microscope at 20×, 5 images per well, to detect HOECHST 33342 (UV lamp, which detects nucleus of all cells), CellTracker™ Green (488 nm laser, which detects target cells only), and propidium iodide (561 nm laser, which detects all dead cells). Images were analyzed using PerkinElmer Columbus software to identify target cells (using CellTracker™ Green intensity) and define them as live or dead based on the intensity of Propidium Iodide stain in the nucleus. The percent dead target cells per well were plotted in GraphPad PRISM software. At 40 hours after adding the BCMA-specific FN3 domain, an aliquot of cells from the original killing assay reaction mixture was collected and stained again and assessed for percent dead target cells. FIG. 4 shows the killing of target cells by TALL-104 CAR-expressing cells in a target specific manner. For AS7B91-CAR-TALL-104 cells at 20 hours after coincubation of cells (FIG. 4A), the killing of RPMI-8226 cells increased in the presence of coincubated 0.32 nM BCMA-specific FN3 domain (40% killing) compared to 10 nM non-targeted control (NT) FN3 domain (27%). The killing of Daudi cells increased from 17% (NT) to 58% in the presence of coincubated 0.32 nM BCMA-specific FN3 domain. Killing of K562 cells did not increase in the presence of coincubated 0.32 nM BCMA-specific FN3 domain. At 40 hours (FIG. 4B), the AS7B91-CAR-TALL-104 cell killing of Daudi cells increased to 70% in the presence of 0.32 nM BCMA-specific FN3 domain from 15% for 10 nM NT FN3 domain. AS7B91-CAR-TALL-104 cell killing of RPMI-8226 cells was 59% in the presence of coincubated 0.32 nM BCMA-specific FN3 domain compared to 45% for NT control. At 40 hours, BCMA-specific killing was not observed in K562 cells. For D08-CAR-TALL-104 cells at 20 hours after coincubation of cells (FIG. 4C), the killing of RPMI-8226 cells was increased (37% killing) compared to Mock TALL-104 cells in the presence of coincubated 0.32 nM BCMA-specific FN3 domain (23%). The killing of Daudi cells increased from 11% (Mock TALL-104) to 42% in the presence of coincubated D08-CAR-TALL-104 cells. The killing of K562 cells increased from 11% (Mock TALL-104) to 20% in the presence of coincubated D08-CAR-TALL-104 cells.

### Example 7: hTERT Engineered TALL-104 Cells with Increased Proliferation Capacity

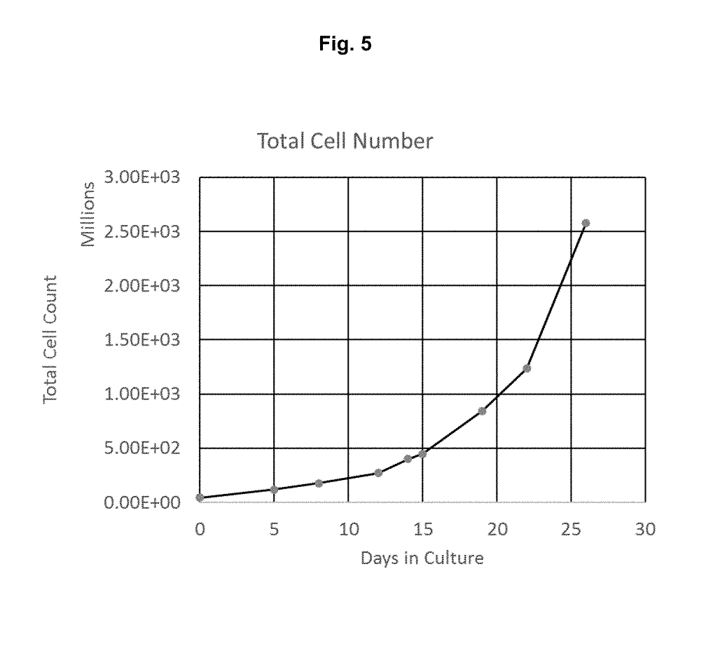

TALL-104 cells were transduced with a lentivirus vector encoding the human TERT gene and an EGFP reporter gene. Green fluorescent cells that were successfully transduced and stably integrated with the transgenes were selected by FACS for EGFP positive cells and allow to expand in TALL-104 media supplemented with human IL-2 according to standard culturing procedures. The cells were allowed to expand over time and continued to expand while non-transduced cells stopped proliferating (FIG. 5).

### Example 8: Engineering of TALL-104 Cells for IL-2 Independent Growth

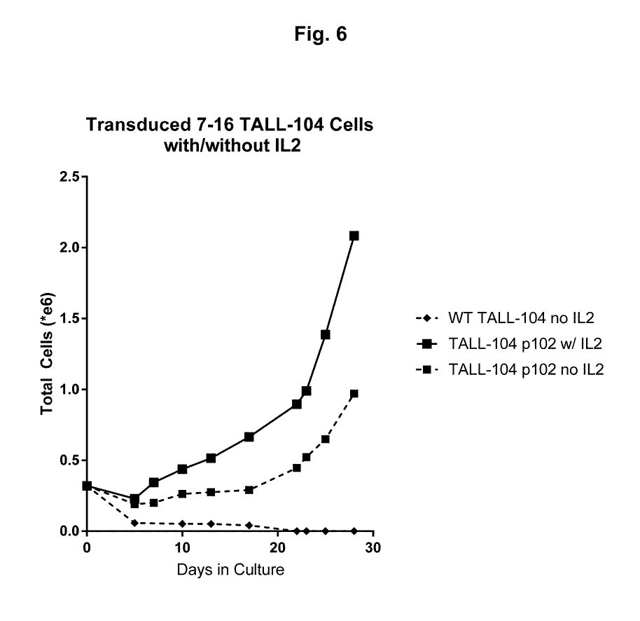

The hTERT transduced cells were further transduced with a second lentivirus vector possessing a human IL-2 transgene with a C-terminal modification, KDEL (SEQ ID NO: 68), which retains the encoded protein in the endoplasmic reticulum of the cells. Culturing of these transduced cells in the absence of exogenous IL-2 resulted in the expansion of successfully transduced cells, while non-transduced cells stopped expanding and died (FIG. 6).

The p102 cells were then tested for targeted killing by transiently electroporating the F11 BCMA targeted CAR mRNA into them. The p102 cells growing in the absence of exogenous IL-2 but stably expressing the ER retained IL-2 kill MM1s cells as effectively as wild-type TALL-104 cells growing with exogenous IL-2 and expressing the same F11 BCMA targeted CAR

We claim:

1. An engineered immortalized T cell line expressing a chimeric antigen receptor (CAR), comprising:
(a) an extracellular domain comprising an antigen binding region;
(b) a transmembrane domain; and
(c) an intracellular signaling domain,
wherein the immortalized T cell line does not express at least one endogenous T cell receptor and does not express beta 2-microglobulin (B2M).

2. The immortalized T cell line of claim 1, wherein the antigen binding region binds a tumor associated antigen.

3. The immortalized T cell line of claim 2, wherein the tumor associated antigen is BCMA.

4. The immortalized T cell line of claim 1, wherein the antigen binding region binds a fibronectin type III (FN3) domain.

5. The immortalized T cell line of claim 1, wherein the at least one endogenous T cell receptor is knocked out.

6. The immortalized T cell line of claim 1, wherein the at least one endogenous T cell receptor is TCR-alpha.

7. The immortalized T cell line of claim 1, wherein the at least one endogenous T cell receptor is KIR3DL2.

8. The immortalized T cell line of claim 1, wherein B2M is knocked out.

9. An engineered TALL-104 cell line expressing a CAR, comprising:
(a) an extracellular domain comprising an antigen binding region;
(b) a transmembrane domain; and
(c) an intracellular signaling domain,
wherein the TALL-104 cell line does not express at least one endogenous T cell receptor and does not express beta 2-microglobulin (B2M).

10. The cell line of claim 9, wherein the antigen binding region binds a tumor associated antigen.

11. The cell line of claim 10, wherein the tumor associated antigen is BCMA.

12. The cell line of claim 9, wherein the antigen binding region binds a fibronectin type III (FN3) domain.

13. The cell line of claim 9, wherein the at least one endogenous T cell receptor is knocked out.

14. The cell line of claim 9, wherein the at least one endogenous T cell receptor is TCR-alpha.

15. The cell line of claim 9, wherein the at least one endogenous T cell receptor is KIR3DL2.

16. The cell line of claim 9, wherein B2M is knocked out.

17. An engineered TALL-104 cell line expressing a CAR, comprising:
(a) a signal peptide having an amino acid sequence of SEQ ID NO: 3;
(b) an extracellular domain comprising an FN3 domain having an amino acid sequence of any one of SEQ ID NOs: 8-44;
(c) a hinge region having an amino acid sequence of SEQ ID NO: 4;
(d) a transmembrane domain having an amino acid sequence of SEQ ID NO: 5; and
(e) an intracellular signaling domain comprising a co-stimulatory domain having an amino acid sequence of SEQ ID NO: 6, and a primary signaling domain having an amino acid sequence of SEQ ID NO: 7;
wherein the cell line does not express TRCA, KIR3DL2 and B2M.

18. An engineered TALL-104 cell line expressing a CAR, comprising:
(a) an extracellular domain comprising an scFv having an amino acid sequence of any one of SEQ ID NOs: 54 and 55;
(b) a hinge region having an amino acid sequence of SEQ ID NO: 4;
(c) a transmembrane domain having an amino acid sequence of SEQ ID NO: 5; and
(d) an intracellular signaling domain comprising a co-stimulatory domain having an amino acid sequence of SEQ ID NO: 6, and a primary signaling domain having an amino acid sequence of SEQ ID NO: 7.
wherein the TALL-104 cell line does not express TRCA, KIR3DL2 and B2M.

19. An in vitro method of generating an engineered immortalized T cell line expressing a CAR, comprising the steps of:
a. providing an immortalized T cell line;
b. inhibiting the expression of at least one endogenous T cell receptor and B2M; and
c. introducing a polynucleotide that encodes a CAR into the immortalized T cell.

20. The method of claim 19, wherein step b occurs before step c.

21. The method of claim 19, wherein step c occurs before step b.

22. The method of claim 19, wherein step b is performed by using an endonuclease.

23. The method of claim 22, where in the endonuclease is a TAL-nuclease, meganuclease, zing-finger nuclease (ZFN), or Cas9.

24. The method of claim 19, wherein step c is further defined as introducing a polynucleotide that encodes a CAR into the immortalized T cell by electroporation or a viral-based gene transfer system.

25. A pharmaceutical composition, comprising the engineered immune cell of any of claims 1, 9, 17 and 18 and a pharmaceutically acceptable carrier.
